(19) United States
(12) Patent Application Publication
(10) Pub. No.: US 2026/0146097 A1
(43) Pub. Date: May 28, 2026

(54) Multi Variable Domain Therapeutic Immunoglobulin

(71) Applicants: Stephen Demarest, San Diego, CA (US); Margaret Karow, Santa Rosa Valley, CA (US); Jianying Dong, San Diego, CA (US); Natasha Del Cid, San Diego, CA (US); Rajesh Sharma, San Diego, CA (US); Matthew Lundberg, Carlsbad, CA (US); Michael Gallo, Vancouver, British Columbia (CA); Jaspal Kang, Surrey, British Columbia (CA); Craig Pigott, Victoria, British Columbia (CA); Abby Yu Chen Lin, Surrey, British Columbia (CA); Tentarix Biotherapeutics, Inc., San Diego, CA (US)

(72) Inventors: Stephen Demarest, San Diego, CA (US); Margaret Karow, Santa Rosa Valley, CA (US); Jianying Dong, San Diego, CA (US); Natasha Del Cid, San Diego, CA (US); Rajesh Sharma, San Diego, CA (US); Matthew Lundberg, Carlsbad, CA (US); Michael Gallo, Vancouver, British Columbia (CA); Jaspal Kang, Surrey, British Columbia (CA); Craig Pigott, Victoria, British Columbia (CA); Abby Yu Chen Lin, Surrey, British Columbia (CA)

(21) Appl. No.: 19/122,505

(22) PCT Filed: Oct. 19, 2023
(86) PCT No.: PCT/US2023/077348

§ 371 Date: Apr. 18, 2025

Related U.S. Application Data
(60) Provisional application No. 63/417,665, filed on Oct. 19, 2022.

(51) Int. Cl. C07K 16/28; A61K 39/00; A61K 45/06

(52) U.S. Cl. CPC C07K 16/2866; A61K 45/06; C07K 16/2803; A61K 2039/505; C07K 2317/31; C07K 2317/52; C07K 2317/565; C07K 2317/75; C07K 2317/76; C07K 2317/92

(57) ABSTRACT

This disclosure relates to an immunoglobulin molecule having multiple variable domains that are each specific for a receptor, as well as heterodimeric molecules that include the multivariable domain immunoglobulin and a second heavy chain constant domain (HCCD2) of an antibody comprising a CH1 and a second Fc region (FC2), where FC1 and FC2 heterodimerize.

### CROSS REFERENCE TO RELATED APPLICATIONS

This application claims the benefit of U.S. Provisional Patent Application No. 63/417,665, filed Oct. 19, 2022, which is incorporated by reference herein in its entirety.

### REFERENCE TO AN ELECTRONIC SEQUENCE LISTING

The contents of the electronic sequence listing (22-1519-WO_ST26_Sequence_Listing.xml; Size: 114,764 bytes; and Date of Creation: Oct. 17, 2023) is herein incorporated by reference in its entirety.

### FIELD OF THE DISCLOSURE

This disclosure generally relates to an immunoglobulin molecule having multiple variable domains that are each specific for a receptor that, in combination, are effective at treating diseases such as cancer.

### BACKGROUND

Cancer is the second-leading cause of death in the world, with 1 in 2 men and 1 in 3 women that will be diagnosed with cancer in their lifetime. Immunotherapy that boosts or changes patients' own immune response to tumor cells is an effective cancer treatment and of great interest. Particularly, drugs targeting the suppressive immuno-checkpoint pathway, such as anti-programmed cell death protein-1 (PD1) inhibitors have shown significant therapeutic benefits by enhancing T-cell function and proliferation. Another checkpoint inhibitor target, lymphocyte-activation gene 3 (LAG3), is upregulated in tumor specific T-cells and other activated immune cells, and shows strong efficacy in combination with PD1 inhibition in metastatic melanoma.

The inventors have identified a need in the art for a multi-functional immunoglobin therapeutic that targets LAG3 and interleukin-2γ/β (IL2Rγ/β) receptors, and thus combines checkpoint inhibition with tumor-targeted interleukin agonism, and is highly specific and efficient in killing tumor cells over a PD1 and LAG3 combination or a PD1/LAG3 bispecific antibody.

### SUMMARY

In various aspects and embodiments, the disclosure is directed to an immunoglobulin molecule having multiple variable domains that are each specific for a receptor, as well as heterodimeric molecules comprising the multi variable domain immunoglobulin and a second heavy chain constant domain (HCCD2) of an antibody comprising a CH1 and a second Fc region (FC2), where FC1 and FC2 heterodimerize.

In another aspect, the disclosure is directed to a single immunoglobulin variable domain that binds to IL-2R gamma (IL2-R gamma VH) including a framework region of a heavy chain variable region of a human antibody and three complimentary determining regions (CDRs), in which CDR1 includes an amino acid sequence selected from SEQ ID NO: 10, SEQ ID NO: 11 or SEQ ID NO: 12; CDR2 includes an amino acid sequence selected from SEQ ID NO: 13, SEQ ID NO: 14 or SEQ ID NO: 15, and CDR3 includes an amino acid sequence selected from SEQ ID NO: 16. In an embodiment, the single immunoglobulin variable domain that binds to IL-2R gamma can include SEQ ID NO: 1.

In certain embodiments of the disclosure, a single immunoglobulin variable domain that binds to IL-2R beta (IL2-R beta VH) is provided, including a framework region of a heavy chain variable region IL2-R beta VH of a human antibody and three complimentary determining regions (CDRs), in which CDR1 includes an amino acid sequence selected from SEQ ID NO: 17, SEQ ID NO:18, SEQ ID NO: 19, SEQ ID NO: 37, SEQ ID NO: 38, or SEQ ID NO: 39; CDR2 includes an amino acid sequence selected from SEQ ID NO: 20, SEQ ID NO:21 or SEQ ID NO: 22; and CDR3 includes an amino acid sequence selected from SEQ ID NO: 23, SEQ ID NO: 40 or SEQ ID NO: 41. Certain examples of such an IL2-R beta VH include SEQ ID NO: 2, SEQ ID NO: 35, SEQ ID NO: 36 or SEQ ID NO: 42.

In another aspect, the disclosure is directed to a multi variable domain agonist including IL2-R gamma VH and IL2-R beta VH, each as described above and herein. Multi variable domain immunoglobulins of the disclosure can include an anti-LAG3 antibody, and IL2-R gamma VH or IL2-R beta VH linked to the N terminal amino acid of a heavy chain of the antibody, which linkage can be serial in nature. In some examples of the multi variable domain immunoglobulin, the IL2-R beta VH is linked to the anti-LAG3 antibody and the IL2-R gamma VH is linked to N-terminus of the IL2-R beta VH; while in other examples, the IL2-R gamma VH is linked to the anti-LAG3 antibody and the IL2-R beta VH is linked to N-terminus of the IL2-R gamma VH.

In some embodiments of the multi variable domain immunoglobulin, the IL2-R beta VH or the IL2-R gamma VH can be linked to the antibody through an amino acid linker, for example, an amino acid linker including 2-20 amino acids. In certain embodiments, the linker between the antibody and the IL2-R beta or gamma VH includes an amino acid sequence including GGQGQGGQGGGQGGG (SEQ ID NO: 61) or PPGGQGQGGQGGGQGGG (SEQ ID NO: 68).

In some embodiments, the IL2-R beta VH is linked to the IL2-R gamma VH through an amino acid linker, for example, an amino acid linker including 2-20 amino acids. In certain embodiments, the linker between the IL2-R beta and gamma VHs includes an amino acid sequence including GGGSGS (SEQ ID NO: 56) or PPGG (SEQ ID NO: 52)

In another aspect, a multi variable domain immunoglobulin including the following an amino acid sequence is provided: [VH1]-[L2]-[VH2]-[L1]-[VH3]-[HCCD1]. In embodiments of this aspect, the HCCD1 includes a first heavy chain constant domain of an antibody including a CH1 and a first Fc region (FC1); VH3 includes a heavy chain variable domain of an anti-LAG3 antibody; VH2 includes one of: (a) a single immunoglobulin variable domain that binds to IL-2R beta (IL2-R beta VH), including a framework region of a heavy chain variable region of a human antibody and three complimentary determining regions (CDRs), in which CDR1 includes an amino acid sequence selected from SEQ ID NO: 17, SEQ ID NO:18, SEQ ID NO: 19, SEQ ID NO: 37, SEQ ID NO: 38, or SEQ ID NO: 39; CDR2 includes an amino acid sequence selected from SEQ ID NO: 20, SEQ ID NO:21 or SEQ ID NO: 22; and CDR3 includes an amino acid sequence selected from SEQ ID NO: 23, SEQ ID NO: 40, or SEQ ID NO: 41; or (b) single immunoglobulin variable domain that binds to IL-2R gamma (IL2-R gamma VH) including a framework region of a heavy chain variable region of a human antibody and three complimentary determining regions (CDRs), in which CDR1 includes an amino acid sequence selected from SEQ ID NO: 10, SEQ ID NO:11 or SEQ ID NO: 12; CDR2 includes an amino acid sequence selected from SEQ ID NO: 13, SEQ ID NO:14 or SEQ ID NO: 15; and CDR3 includes an amino acid sequence selected from SEQ ID NO: 16; VH1 includes the other of the IL2-R beta VH or IL2-R gamma VH; and L1 and L2 are independently optional and may be the same or different amino acid sequence including 2-20 amino acids.

In certain multi variable domain immunoglobulin embodiments of the disclosure, VH2 is the IL2-R gamma VH, and VH1 is the IL2-R beta VH, while in other embodiments, VH2 is the IL2-R beta VH, and VH1 is the IL2-R gamma VH. In additional examples of such multi variable domain immunoglobulins, the VH2 includes SEQ ID NO: 2, SEQ ID NO: 35, SEQ ID NO:36 or SEQ ID NO:42; and in some examples, the VH1 includes SEQ ID NO: 1. In further examples, one of either (a) the FC1 includes SEQ ID NO: 44, SEQ ID NO: 45, SEQ ID NO: 46, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, or SEQ ID NO: 116; or (b) HCCD1 includes SEQ ID NO: 4, SEQ ID NO: 5, SEQ ID NO: 43, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, or SEQ ID NO: 105.

In some embodiments of multi variable domain immunoglobulins with the arrangement [VH1]-[L2]-[VH2]-[L1]-[VH3]-[HCCD1], the VH3 includes a framework region of a heavy chain variable region of anti-LAG3 antibody and three complimentary determining regions (CDRs), in which CDR1 includes an amino acid sequence selected from SEQ ID NO: 24, SEQ ID NO: 25 or SEQ ID NO: 26; CDR2 includes an amino acid sequence selected from SEQ ID NO: 27, SEQ ID NO: 28 or SEQ ID NO: 29; and CDR3 includes an amino acid sequence selected from SEQ ID NO: 30. In additional embodiments, the VH3 includes SEQ ID NO: 3.

In some embodiments, the multi variable domain immunoglobulin described above and herein further including a light chain variable domain of the anti-LAG3-antibody (VL1), which VL1, in some embodiments, includes a framework region of alight chain variable region of an anti-LAG3 antibody and three complimentary determining regions (CDRs), in which CDR1 includes SEQ ID NO: 31; CDR2 includes SEQ ID NO: 32; and CDR3 includes an amino acid sequence selected from SEQ ID NO: 33, or SEQ ID NO: 34. In some embodiments, the VL1 includes SEQ ID NO: 6 or SEQ ID NO: 8.

In some embodiments, the multi variable domain immunoglobulin described above and herein further including a light chain including an amino acid sequence selected from SEQ ID NO: 7 or SEQ ID NO: 9.

In some embodiments of multi variable domain immunoglobulin, L1 and L2 are independently selected from an amino acid sequence including PP, GG, PPGG (SEQ ID NO: 52), GGGSGS (SEQ ID NO:56); GGQGQGGQGGGQGGG (SEQ ID NO: 61), or PPGGQGQGGQGGGQGGG (SEQ ID NO: 68), preferably wherein linker L1 includes PP, GG, PPGG (SEQ ID NO:52), or GGGSGS (SEQ ID NO: 56), and L2 includes PP, GGQGQGGQGGGQGGG (SEQ ID NO: 61), or PPGGQGQGGQGGGQGGG (SEQ ID NO: 68).

In another aspect, the disclosure provides a heterodimeric molecule including a multi variable domain immunoglobulin as described above and herein and a second heavy chain constant domain (HCCD2) of an antibody including a CH1 and a second Fc region (FC2), wherein FC1 and FC2 heterodimerize.

In some embodiments of the heterodimeric molecule, either (a) HCCD1 includes SEQ ID NO: 5, SEQ ID NO: 97, SEQ ID NO: 99, SEQ ID NO: 101, SEQ ID NO: 103, or SEQ ID NO: 105, when HCCD2 includes SEQ ID NO:4, SEQ ID NO: 96, SEQ ID NO: 98, SEQ ID NO: 100, SEQ ID NO: 102, or SEQ ID NO: 104, respectively; or (b) HCCD2 includes SEQ ID NO: 4, SEQ ID NO: 96, SEQ ID NO: 98, SEQ ID NO: 100, SEQ ID NO: 102, or SEQ ID NO: 104 when HCCD1 includes SEQ ID NO:5, SEQ ID NO: 97, SEQ ID NO: 99, SEQ ID NO: 101, SEQ ID NO: 103, or SEQ ID NO: 105, respectively.

Some embodiments of the heterodimeric molecule further include a VH3 linked to the N-terminus of HCCD2, and some embodiments further including a light chain variable domain of the anti-LAG3-antibody (VL1), which VL1 can, in some embodiments, include SEQ ID NO: 6 or SEQ ID NO: 8.

Some embodiments of the heterodimeric molecule further including a light chain including an amino acid sequence selected from SEQ ID NO: 7 or SEQ ID NO:9.

In another aspect, the disclosure provides a heterodimeric molecule including (a) a first heavy chain constant domain (HCCD1) of an antibody including a CH1 and a first Fc region; (b) an anti-LAG3 VH (VH3) linked to the CH1; (c) a second Fc region (FC2) that heterodimerizes with FC1; (d) a VH1 including a single immunoglobulin variable domain that binds to IL-2R gamma, including a framework region of a heavy chain variable region of a human antibody and three complimentary determining regions (CDRs), in which CDR1 includes an amino acid sequence selected from SEQ ID NO: 10, SEQ ID NO:11 or SEQ ID NO: 12; CDR2 includes an amino acid sequence selected from SEQ ID NO: 13, SEQ ID NO:14 or SEQ ID NO: 15; and CDR3 includes an amino acid sequence selected from SEQ ID NO: 16; (e) a VH2 including single immunoglobulin variable domain that binds to IL-2R beta including a framework region of a heavy chain variable region of a human antibody and three complimentary determining regions (CDRs), in which CDR1 includes an amino acid sequence selected from SEQ ID NO: 17, SEQ ID NO:18, SEQ ID NO: 19, SEQ ID NO: 37, SEQ ID NO: 38, or SEQ ID NO: 39; CDR2 includes an amino acid sequence selected from SEQ ID NO: 20, SEQ ID NO:21 or SEQ ID NO: 22; and CDR3 includes an amino acid sequence selected from SEQ ID NO: 23, SEQ ID NO: 41, or SEQ ID NO: 41; and (f) a VH3 including a framework region of a heavy chain variable region of anti-LAG3 antibody and three complimentary determining regions (CDRs), in which CDR1 includes an amino acid sequence selected from SEQ ID NO: 24, SEQ ID NO: 25 or SEQ ID NO: 26; CDR2 includes an amino acid sequence selected from SEQ ID NO: 27, SEQ ID NO: 28 or SEQ ID NO: 29; and CDR3 includes an amino acid sequence selected from SEQ ID NO: 30; wherein VH1 and VH2 are serially linked to the N-terminus of the FC2, and the VH3 is linked to the N-terminus of the CH1. In some embodiments, the VH2 is linked to FC2 and the VH1 is linked to an N-terminus of the VH2; while, in some embodiments, the VH1 is linked FC2 and the VH2 is linked to an N-terminus of the VH1.

In some embodiments of the heterodimeric molecule, the VH1 includes SEQ ID NO: 1; and in some embodiments, the VH2 includes SEQ ID NO: 2, SEQ ID NO: 35, SEQ ID NO: 36 or SEQ ID NO: 42. In some embodiments, the VH3 includes SEQ ID NO: 3.

In additional embodiments of the heterodimeric molecule, the HCCD1 includes SEQ ID NO: 4, SEQ ID NO:5, SEQ ID NO: 96, SEQ ID NO: 97, SEQ ID NO: 98, SEQ ID NO: 99, or SEQ ID NO: 100, SEQ ID NO: 101, SEQ ID NO: 102, SEQ ID NO: 103, SEQ ID NO: 104, or SEQ ID NO: 105. Further embodiments include either (a) FC2 including SEQ ID NO: 45, SEQ ID NO: 108, SEQ ID NO: 110, SEQ ID NO: 112, SEQ ID NO: 114, or SEQ ID NO: 116, when HCCD1 includes SEQ ID NO:4, SEQ ID NO: 96, SEQ ID NO: 98, SEQ ID NO: 100, SEQ ID NO: 102, or SEQ ID NO: 104, respectively; or (b) FC2 including SEQ ID NO: 44, SEQ ID NO: 107, SEQ ID NO: 109, SEQ ID NO: 111, SEQ ID NO: 113, or SEQ ID NO: 115, when HCCD1 includes SEQ ID NO: 5, SEQ ID NO: 97, SEQ ID NO: 99, SEQ ID NO: 101, SEQ ID NO: 103, or SEQ ID NO: 105, respectively.

Some embodiments of the heterodimeric molecule further include a light chain variable domain of the anti-LAG3-antibody (VL1), which VL1, in some embodiments, includes SEQ ID NO: 6 or SEQ ID NO: 8, and some embodiments of the heterodimeric molecule further include a light chain including an amino acid sequence selected from SEQ ID NO: 7 or SEQ ID NO: 9.

In some embodiments of the heterodimeric molecule, the VH1 or VH2 is linked to the FC2 through an amino acid linker, for example an amino acid linker including 2-20 amino acids, for example linker is selected from GG, PP, PPGG, (SEQ ID NO: 52), GGGSGS (SEQ ID NO: 56), GGQGQGGQGGGQGGG (SEQ ID NO: 61), or PPGGQGQGGQGGGQGGG (SEQ ID NO: 68). In some embodiments, the VH1 is linked to the VH2 through an amino acid linker, for example an amino acid linker including 2-20 amino acids, for example linker is selected from GG, PP, PPGG, (SEQ ID NO: 52), GGGSGS (SEQ ID NO: 56), GGQGQGGQGGGQGGG (SEQ ID NO: 61), or PPGGQGQGGQGGGQGGG (SEQ ID NO: 68).

In another aspect, a multi variable domain immunoglobulin is provided including SEQ ID NO 48, SEQ ID NO: 49, SEQ ID NO: 50, SEQ ID NO: 51, SEQ ID NO: 86, SEQ ID NO: 87, SEQ ID NO: 89, SEQ ID NO: 90, SEQ ID NO: 91, SEQ ID NO: 92, SEQ ID NO: 93, SEQ ID NO: 94, or SEQ ID NO: 95.

In another aspect, heterodimeric molecules are provided, including one comprising SEQ ID NO: 9, SEQ ID NO: 48, and SEQ ID NO: 49; another heterodimeric molecule includes SEQ ID NO: 9, SEQ ID NO: 50, and SEQ ID NO: 49; another includes SEQ ID NO: 9, SEQ ID NO: 51, and SEQ ID NO: 49; another includes SEQ ID NO: 9, SEQ ID NO: 86, and SEQ ID NO: 87; another includes SEQ ID NO: 9, SEQ ID NO: 89, and SEQ ID NO: 90; another includes SEQ ID NO: 9, SEQ ID NO: 91, and SEQ ID NO: 92; another includes SEQ ID NO: 9, SEQ ID NO: 93, and SEQ ID NO: 92; and another includes SEQ ID NO: 9, SEQ ID NO: 94, and SEQ ID NO: 95.

In another embodiment, heterodimeric molecules are provided including SEQ ID NO: 9, and one of SEQ ID NO: 48, SEQ ID NO: 50, SEQ ID NO: 51, SEQ ID NO: 86, SEQ ID NO: 89, SEQ ID NO: 91, SEQ ID NO: 93, or SEQ ID NO: 94, and one of SEQ ID NO: 49, SEQ ID NO: 87, SEQ ID NO: 90, SEQ ID NO: 92, or SEQ ID NO: 95.

In another aspect, the dislosure provides a polynucleotide encoding the single immunoglobulin variable domain or the heterodimeric molecule as described above and herein.

In another aspect, the disclosure provides a pharmaceutical composition, including the single immunoglobulin variable domain or the heterodimeric molecule as described above and herein. In some embodiments, the pharmaceutical composition further including a PD1 inhibitor.

### BRIEF DESCRIPTION OF THE FIGURES

The following detailed description of the embodiments of the present invention can be best understood when read in conjunction with the following drawings, where like structure is indicated with like reference numerals and in which:

FIG. 1 shows a schematic diagram of an example embodiment of the disclosure designated TNRX-257.

FIG. 2 shows a schematic diagram of an example embodiment of the disclosure designated TNRX-190.

FIG. 3 shows analytical hydrophobic interaction chromatography (HIC) chromatograms of purified control proteins Trastuzumab IgG1 and Urelumab IgG1 and anti-IL2R-VH-Fc proteins with wild-type HCDR3 with a proline at position 100c (Kabat) and two variants P100cS and P100cT.

FIG. 4 shows (ELISA) binding of soluble LAG3-histag-biotin to wild-type LAG3i-IL2Rgamma/beta multi-specific, as well as multiple single amino acid variants within the light chain LCDR3 that improved binding.

FIG. 5A shows a two-step preparative purification of TNRX-257 (panel (A) shows protein A capture, and panel (B) shows preparative size exclusion chromatography (SEC)).

FIG. 5B shows analytical size exclusion chromatography (panel (C)) and reduced sodium dodecyl sulfate polyacrylamide gel electrophoresis (SDS-PAGE; panel (D)) characterization of TNRX-257.

FIG. 6A shows a two-step preparative purification of TNRX-190 (panel (A) shows protein A capture, and panel (B) shows preparative size exclusion chromatography (SEC)).

FIG. 6B shows analytical size exclusion chromatography (panel (C)) and reduced sodium dodecyl sulfate polyacrylamide gel electrophoresis (SDS-PAGE; panel (D)) characterization of TNRX-190.

FIG. 7 shows blockade of LAG3-Fc binding to MHC class II expressing Daudi cells by TNRX-257.

FIG. 8 shows detection of TNRX-257-mediated LAG3 inhibition of T cell receptor suppression in an NFAT reporter luminescence assay.

FIG. 9A shows IL2R activation measured by STAT5 phosphorylation on LAG3+ CD8+ T cells. FIG. 9B shows IL2R activation measured by STAT5 phosphorylation on LAG3− CD8+ T cells.

FIG. 10A shows evaluation of the expansion of LAG3+ CD8+ T cells from a metastatic melanoma biopsy using CTV staining. FIG. 10B shows evaluation of the expansion of LAG3− CD8+ T cells from a metastatic melanoma biopsy using CTV staining.

FIG. 11 shows the measurement of IFNgamma release from melanoma TILs cultured in the absence (no stimulation) and presence of TNRX-257, LAG3 inhibitor mAb, control IgG, or an IL15/IL15R-Fc untargeted IL2Rgamma/beta agonist in the absence or presence of a saturating level of pembrolizumab (anti-PD1).

FIG. 12 shows the change in tumor volume over time in an in vivo A375-CMV+ melanoma tumor model in NSG MHC DKO mice injected on day 0 with 10M PBMCs from a CMV+/HLA-matched donor. Dosing was initiated on day 4 after tumor cell implantation.

FIG. 13 shows the change in tumor volume over time in an in vivo A375-CMV+ melanoma tumor model in NSG MHC DKO mice injected on day 0 with 10M PBMCs from a CMV+/HLA-matched donor. Dosing was initiated on day 13 after tumor cell implantation.

### DESCRIPTION

The disclosure is directed to immunoglobulin molecules and pharmaceutical compositions for treating diseases such as cancer. The molecules of the disclosure include antibody VH domains directed IL2Rγ or IL2Rβ, alone or in combination with each other or with a VH domain from an anti-LAG3 antibody.

When in combination, the anti-LAG3 domain in LAG3 Inhibitor-IL2Rγ/β Agonist (“LAG3i/IL2R”) molecule inhibits LAG3 receptor, while the IL2Rγ and IL2Rβ domains at least partially agonize IL2 and IL15 receptors. IL2 and IL15 receptor activation induces anti-tumor activity by promoting proliferation and cytokine production in T cells and NK cells. Meanwhile, binding of LAG3i-IL2R molecule to LAG3 receptor prevents MHC class II molecules from binding and activating LAG3 downstream signaling. Blocking LAG3 signaling releases the inhibition on T cell receptor signaling/function and dampens the immunosuppressive regulatory T cells (Treg) function. Thus, a LAG3i-IL2R molecule's function is two-fold: inhibiting the immune suppressor and upregulating the immune activator, to tip the balance of the immune response towards anti-tumor activity.

The LAG3i-IL2R molecules of the disclosure have higher potency IL2R agonism on LAG3 expressing cells (LAG3+) than on non-LAG3 expressing cells (LAG3−) and thus, the IL2R agonist activity of LAG3i-IL2R molecule is guided towards LAG3+ cells. Immune cell populations with LAG3+ cells include 1) antigen-activated T cells and tumor-specific T cells, 2) Fc gamma receptor (FcgR) some activated subpopulations of NK cells and macrophages 3) some B cells, and 4) some plasmacytoid dendritic cells (pDCs). Since LAG3 is a marker of tumor reactive T cells within the tumor microenviroment (Gros A et al., 2014 J Clin Invest 124(5):2246-59) inhibiting LAG3-mediated TCR suppression and activation of IL2Rgamma/IL2Rbeta specifically on these cells should increase their anti-tumor activity. For examples, IL2 agonist activity of a LAG3i-IL2R molecule is only functional in antigen-activated T cells which have LAG3 on their cell surface. Consistent with this, only LAG3+ T cells in the tumor infiltrating lymphocyte population (TIL) are activated, proliferate and express IFNgamma when stimulated with the LAG3i-IL2R molecule. Additionally, directing LAG3i-IL2R molecule to LAG3+ cells directs the activity to tumor-specific cells, and avoids the activity in the blood and normal tissues that can be dose limiting for IL2 and IL15 cytokine based therapeutics

Due to its specificity to LAG3+ immune cells, LAG3i-IL2R molecule may be administered in combination with other cancer drugs in the market that upregulate and/or require LAG3+ immune cells to kill tumor cells. Other cancer drugs (pembrolizumab, nivolumab and cemiplimab-rwlc) targets and inhibits PD1 which is another immunosuppressive checkpoint pathway similar to LAG3. Anti-PD1 treatment has been shown to upregulate LAG3 expression in CD8+ T cells. Furthermore, IL2R partial agonists expand the stem-like effector population of CD8+ T cells that are enriched with PD1. Thus, combination therapy of LAG3i-IL2R molecule and anti-PD1 drugs can potentially provide a synergistic effect in activating more anti-tumor immune cells.

Before describing the various aspects of the disclosure, a number of terms will be defined. Unless otherwise required by context, singular terms shall include pluralities and plural terms shall include the singular. For example, the singular forms “a,” “an,” and “the” include plural referents unless the context clearly dictates otherwise.

It is noted that terms like “preferably,” “commonly,” and “typically” are not utilized herein to limit the scope of the disclosure or to imply that certain features are critical, essential, or even important to the structure or function of the disclosure. Rather, these terms are merely intended to highlight alternative or additional features that can or cannot be utilized in a particular embodiment of the disclosure. A patent literature cited herein is incorporated by reference herein in its entirety.

For the purposes of describing and defining the various aspects of the disclosure it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As utilized in accordance with the present disclosure, unless otherwise indicated, all technical and scientific terms shall be understood to have the same meaning as commonly understood by one of ordinary skill in the art.

The term “amino acid” or “residue” as used within this application denotes the group of naturally occurring carboxy α-amino acids including alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “immunoglobulin” refers to a protein having the structure of a naturally occurring antibody, as described hereinbefore.

An “antibody” refers to a glycoprotein including at least two heavy (H) chains and two light (L) chains inter-connected by disulfide bonds and having a structure substantially similar to a native antibody structure. For example, native IgG-class antibodies are heterotetrameric glycoproteins of about 150 kilodaltons (kD), composed of two light chains and two heavy chains that are disulfide-bonded. From N- to C-terminus, each heavy chain has a variable region (VH), followed by three constant domains (CH1, CH2, and CH3) (also called a heavy chain constant region). Similarly, from N- to C-terminus, each light chain has a variable region (VL) followed by a light chain constant domain (CL) (also called a light chain constant region). The heavy chain of an antibody may be assigned to one of five types, called α (IgA), δ (IgD), ε (IgE), γ (IgG), or μ, (IgM), some of which may be further divided into subtypes, e.g., γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain including four conserved framework regions (FRs) and three hypervariable regions (HVRs). A single VH or VL domain may be sufficient to confer antigen-binding specificity, although the disclosure herein is focused on VH domains.

The term “Tentacle” as used herein refers to multifunctional biologics that are targeted, synergistic and conditional, where “targeted” refers to cell surface proteins that enable selectivity with desired function, where “synergistic” refers to cell surface proteins that, when combined, act synergistically, and “conditional” refers to enabling biological activity in a cell when all targets are engaged. Tentacles can activate a target cell population when all desired receptors are present.

The term “complementarity determining region(s)” or “CDR(s)” as used herein refers to each of the regions of an antibody variable domain which are hypervariable in sequence and/or form structurally defined loops (“hypervariable loops”) and/or contain the antigen-contacting residues (“antigen contacts”). Generally, antibodies include six CDRs: three in the VH (HCDR1, HCDR2, HCDR3), and three in the VL (LCDR1, LCDR2, LCDR3). “Kabat numbering” for CDRs refers to the numbering system set forth by Kabat et al., U.S. Dept. of Health and Human Services, “Sequence of Proteins of Immunological Interest” (1983). Over the years, a number of different approaches have been used to identify the CDR amino sequences within VH and VL sequences. In view of the different approaches, CDRs within a single VH or VL sequence may be identified as having different but overlapping sequences, or having sequences that are shorter or longer, depending on the method used for identifying the CDRs.

In one known approach, “Kabat numbering” for CDRs refers to the numbering system set forth by Kabat et al., U.S. Dept. of Health and Human Services, “Sequence of Proteins of Immunological Interest” (1983). Using this approach, CDR residues and other residues in the variable domain (e.g., FR residues) are numbered herein “the Kabat numbering system” to assign a position to any variable region sequence, without reliance on any experimental data beyond the sequence itself.

Another approach to CDR numbering is “Chothia numbering,” which to a CDR numbering system identified in Chothia C, Lesk a M. (Canonical structures for the hypervariable regions of immunoglobulins. J Mol Biol. (1987) 196:901-17).

Yet another approach to CDR numbering, known as “Martin numbering,” is the described in Martin Abhinandan K R, Martin A C R. Analysis and improvements to Kabat and structurally correct numbering of antibody variable domains. (Mol Immunol. (2008) 45:3832-9).

The following table reflects a comparison between the CDR numbering using the three above described approaches:

| CDR | Kabat | Chothia | Martin | |
|---|---|---|---|---|
| L1 | 24-34 | 24-34 | 24-34 | |
| L2 | 50-56 | 50-56 | 50-56 | |
| L3 | 89-97 | 89-97 | 89-97 | |
| H1 | 31-35 | 26-32 | 26-35 | (K + C) |
| H2 | 50-65 | 52-56 | 50-58 | (K − 7) |

| | | | |
|---|---|---|---|
| H3 | 95-102 | 95-102 | 95-102 |

When the Kabat, Chothia and Martin numbering systems identify different CDRs within a single VH or VL, this disclosure identifies each of the CDRs. In addition, the disclosure includes CDR regions that would include each of the CDRs, regardless of the numbering system selected. For example, a CDR1 region would include all of the amino acids that would appear in the sequences identified by any of the numbering systems, although a CDR region may include additional amino acids at either terminus of a particular CDR if any numbering system would include those additional amino acids. Accordingly, as an example of one aspect of the disclosure, a CDR1 region would include the CDR1 sequence identified by any of the Kabat, Chothia or Martin systems. Similarly, a CDR2 region would include a CDR2 sequence identified by any one of the systems and a CDR3 region would include a CDR3 sequence identified by any one of the systems.

The polypeptide sequences of the Sequence Listing herein are not numbered according to any numbering system. However, it is well within the ordinary skill of one in the art to convert the numbering of the sequences of the Sequence Listing to an appropriate numbering system.

“Framework” or “FR” refers to variable domain residues other than CDR residues. The FR of a variable domain generally consists of four FR regions: FR1, FR2, FR3, and FR4. Accordingly, the CDR and FR sequences generally appear in the following sequence either a VH or VL: FR1-CDR1-FR2-CDR2-FR3-CDR3-FR4. For simplicity in the context of the VH domains described herein, references to FR1, FR2, FR3 and FR4 are intended to refer the FR regions of the VH domains (with the understanding that VL domains also have FRs).

As used herein, the term “Fc region” refers to the portion of an immunoglobulin formed by two or more Fc moieties of antibody heavy chains. In certain embodiments, the Fc region is a single chain that dimerizes with a second chain to form dimeric Fc region. A “dimeric Fc region” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties. As used herein “Fc region” may refer to either a single chain or dimeric form depending on the context.

In certain embodiments, an Fc moiety comprises at least one of: a hinge (e.g., upper, middle, and/or lower hinge region) domain, a CH2 domain, a CH3 domain, or fragment thereof. In preferred embodiments, an Fc moiety comprises at least a CH2 domain or a CH3 domain. In certain embodiments, the Fc moiety is a complete Fc moiety.

As used herein, a heavy chain constant domain (HCCD) includes an Fc region and a CH1 region connected to a hinge domain of the Fc region. In other words the HCCD includes the heavy chain constant region (CH3, CH2) and the CH1 connected at the hinge domain.

In other embodiments, the Fc moiety comprises one or more amino acid insertions, deletions, or substitutions relative to a naturally-occurring Fc moiety. For example, at least one of a hinge domain, CH2 domain or CH3 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a CH2 domain (or portion thereof), and (v) a CH3 domain or portion thereof.

As set forth herein, it will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. For example, the Fc moiety may comprise or consist of at least the portion of an Fc moiety that is known in the art to be required for FcRn binding or extended half-life. In another embodiment, an Fc moiety comprises at least the portion known in the art to be required for FcγR and C1q binding. In one embodiment, an Fc region of the invention comprises at least the portion of known in the art to be required for Protein A binding. In one embodiment, an Fc moiety of the invention comprises at least the portion of an Fc molecule known in the art to be required for protein G binding.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms.

The term “nucleic acid molecule” or “polynucleotide” includes any compound and/or substance that includes a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers including two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides.

An “isolated” nucleic acid molecule or polynucleotide refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The terms “pharmaceutical composition” or “therapeutic composition” as used herein refer to a compound or composition capable of inducing a desired therapeutic effect when properly administered to a patient. In some embodiments, the disclosure provides a pharmaceutical composition including a pharmaceutically acceptable carrier and a therapeutically effective amount of immunotoxin fusion proteins of the disclosure.

The terms “pharmaceutically acceptable carrier” or “physiologically acceptable carrier” as used herein refer to one or more formulation materials suitable for accomplishing or enhancing the delivery of one or more heavy chain variable domains of the disclosure.

Turning now to the various aspects of the disclosure, the inventors have identified a number of approaches construct single chains and dimeric molecules that provide a multi-functional immunoglobulin therapeutics that targets LAG3 and interleukin-2γ/β (IL2Rγ/β) receptors.

### Multi Variable Domain Agonists

In one aspect, the disclosure is directed to a single immunoglobulin variable domain that binds to IL-2R gamma (which is designated in some aspects herein as “IL2-R gamma VH”). ILR-R gamma VH, which includes a framework region of a heavy chain variable region of a human antibody and following three complimentary determining regions (CDRs):

| | Numbering | | SEQ |
|---|---|---|---|
| CDR | System* | Sequence | ID NO: |
| 1 | M | GFTFDDYGMS | 10 |
| 1 | K | DYGMS | 11 |
| 1 | C | GFTFDDY | 12 |
| 2 | M | GINWNGGST | 13 |
| 2 | K | GINWNGGSTGYADSVK | 14 |
| 2 | C | NWNG | 15 |
| 3 | M, K, C | ATSGTAFDI | 16 |

*K = Kabat, C = Chothia, M = Martin

All of these CDRs are contained in the framework region that provides the complete IL2-R gamma VH having SEQ ID NO: 1 as follows:

(SEQ ID NO: 1)
EVQLVESGGGVVRPGGSLRLSCCASGFTFDDYGMSWVRRAPG
KGLEWVAGINWNGGSTGYADSVKGRFTISRDNEKNCLYLQMN
SLRAEDTALYHCASATSGTAFDIWGQGTMVTVSS

Underlined portions of the sequence reflect the regions of the VH that contain the amino acids for each CDR under any of the numbering systems.

Another aspect of the disclosure is directed to a single immunoglobulin variable domain that binds to IL-2R beta (which is designated in some aspects herein as “IL2-R beta VH”). The ILR-R beta VH includes a framework region of a heavy chain variable region of a human antibody and following three complimentary determining regions (CDRs):

| | Numbering | | SEQ ID |
|---|---|---|---|
| CDR | System | Sequence | NO: |
| 1 | M | GYTFTRYYMH | 17 |
| 1 | K | RYYMH | 18 |
| 1 | C | GYTFTRY | 19 |
| 2 | M | LVDPEDGE | 20 |
| 2 | K | LVDPEDGETIYAEKFQ | 21 |
| 2 | C | DPEDG | 22 |
| 3 | M, K, C | DFVKLDESLSFQH | 23 |

All of these CDRs are contained in a framework region that provides the complete IL2-R beta VH having SEQ ID NO: 2 as follows:

(SEQ ID NO: 2)
EVQLVQSGAEVKKPGACVKISCKVSGYTFTRYYMHWVQRAPGKGLEW
IGLVDPEDGETIYAEKFQGRVTITADTSTDTAYMELCSLRSEDTAVY
YCATDFVKLDESLSFQHWGQGTLVTVSS

Underlined portions of the sequence reflect the regions of the VH that contain the amino acids for each CDR under any of the numbering systems.

The IL2-R beta VH of SEQ ID NO: 2 includes a mutation P108S that that has been introduced to increase hydrophobicity the of the VH. Accordingly, the WT P108 sequence is reflected in SEQ ID NO: 35 as shown here:

(SEQ ID NO: 35)
EVQLVQSGAEVKKPGACVKISCKVSGYTFTRYYMHWVQRAPGKGLEW
IGLVDPEDGETIYAEKFQGRVTITADTSTDTAYMELCSLRSEDTAVY
YCATDFVKLDFSLPFQHWGQGTLVTVSS

In another embodiment, the mutation at position P108 is P108T as shown in SEQ ID NO: 36:

(SEQ ID NO: 36)
EVQLVQSGAEVKKPGACVKISCKVSGYTFTRYYMHWVQRAPGKGLEW
IGLVDPEDGETIYAEKFQGRVTITADTSTDTAYMELCSLRSEDTAVY
YCATDFVKLDESLTFQHWGQGTLVTVSS

Accordingly, in view of the possible mutants to the IL2-R beta VH at position 108a, CDR3 may be any of DFVKLDFSLSFQH (SEQ ID NO: 23), DFVKLDFSLPFQH (SEQ ID NO: 40), or DFVKLDFSLTFQH (SEQ ID NO: 41).

In addition to the P108S/T mutation in SEQ ID NO: 2, the CDR1 of the IL-R beta VH of SEQ ID NO:2 reflects an affinity matured (D to R) variant that was to increase receptor binding affinity. The resulting CDRs, without the variant, of another IL2-R beta VH of the disclosure are as follows:

| | Numbering | | SEQ ID |
|---|---|---|---|
| CDR | System* | Sequence | NO: |
| 1 | M | GYTFTDYYMH | 37 |
| 1 | K | DYYMH | 38 |
| 1 | C | GYTFTDY | 39 |
| 2 | M | LVDPEDGE | 20 |
| 2 | K | LVDPEDGETIYAEKFQ | 21 |
| 2 | C | DPEDG | 22 |
| 3 | M, K, C | DFVKLDFSLSFQH | 23 |

All of these CDRs are contained in a framework region that provides the complete IL2-R beta VH having SEQ ID NO: 42 as follows:

(SEQ ID NO: 42)
EVQLVQSGAEVKKPGACVKISCKVSGYTFTDYYMHWVQRAPGKG
LEWIGLVDPEDGETIYAEKFQGRVTITADTSTDTAYMELCSLRS
EDTAVYYCATDFVKLDESLSFQHWGQGTLVTVSS

In one aspect, the disclosure is directed to a multi variable domain agonist that includes the IL2-R gamma VH having the CDRs or the complete VH sequence as described herein the IL2-R beta VH having the CDRs or the complete VH sequence as described herein. The VH's may be linked with an optional linker of 2-20 amino acids as further described herein.

In another aspect, the disclosure is directed to a multi variable domain immunoglobulin including an anti-LAG3 antibody and the IL2-R gamma VH or the IL2-R beta VH linked to the N-terminus of a heavy chain of the antibody. The IL2-R gamma VH or the IL2-R beta VH may be linked to the N-terminus of the antibody through an amino acid linker, for example a 2-20 amino acid linker as further described hererin. In another aspect, the disclosure is directed to an anti-LAG3 antibody and the IL2-R gamma VH and the IL2-R beta VH linked to the N-terminus of a heavy chain of the antibody. In this aspect, either (a) the IL2-R beta VH is linked to the anti-LAG3 antibody and the IL2-R gamma VH is linked to N-terminus of the IL2-R beta VH, or (b) the IL2-R gamma VH is linked to the anti-LAG3 antibody and the IL2-R beta VH is linked to N-terminus of the IL2-R gamma VH. Each of these linkages may include the 2-20 amino acid linker as further described herein. Particular non-limiting embodiments of the linker between the IL2 HVs and the linker between either of the IL-2 VHs are shown below and further described herein.

### Anti-LAG3 Antibodies

The anti-LAG3 antibody appropriate for the molecules of the disclosure may be selected from any one of several know antibodies. For instance, the anti-LAG3 VH and VL sequences are taken from the anti-LAG3 antibody known as 26H10. Table 1 shows a number of other anti-LAG3 antibodies that would be useful in the multi variable domain immunoglobulins or chimeric molecules of the disclosure.

TABLE 1

| Company | Generic Drug Name | Targets |
|---|---|---|
| Bristol-Myers Squibb | relatlimab | Anti-LAG3 |
| Regeneron Sanofi | fianlimab | Anti-LAG3 |
| Merck & Co. | favezelimab | Anti-LAG3 |
| Boehringer Ingelheim | BI-754111 | Anti-LAG3 |
| Zhejiang Shimai | DNV-3 | Anti-LAG3 |
| Innovent Biologics | IBI-110 | Anti-LAG3 |
| Agenus/Incyte | INCAGN-2385 | Anti-LAG3 |
| Leads Biolabs | LBL-007 | Anti-LAG3 |
| Immutep | leramilimab | Anti-LAG3 |
| Roche | RO-7247669 | PD1 × LAG3 |
| Servier Takeda | Sym-022 | Anti-LAG3 |
| Shanghai Fosun | HLX-26 | Anti-LAG3 |
| Innovent Biologics | IBI-323 | PDL1 × LAG3 |
| Sichuan Kelun | KL-A289 | Anti-LAG3 |
| AnaptysBio | TSR-033 | Anti-LAG3 |
| GlaxoSmithKline | | |
| ABL Bio | ABL-401 | Anti-LAG3 |
| Harbin Gloria | anti-LAG3 antibody | Anti-LAG3 |
| Pharmaceuticals | | |
| Elpiscience | ES-005 | Anti-LAG3 |
| ImmuneOncia Therapeutics | IMC-003 | Anti-LAG3 |
| Beijing Mabworks Biotech | MIL-98 | Anti-LAG3 |
| REMD Biotherapeutics | REMD-813 | PD1 × LAG3 |
| AnaptysBio | TSR-075 | PD1 × LAG3 |
| GlaxoSmithKline | | |
| Y-Biologics | YBL-011 | Anti-LAG3 |
| Xencor | XmAb-841 | CTLA4 × LAG3 |
| Eli Lilly | AM-0003 | Anti-LAG3 |
| 3SBio | 619 | Anti-LAG3 |
| ABL Bio | ABL-50X | PDL1 × LAG3 |
| Novartis | anti LAG3 antbody, | Anti-LAG3 |
| | Novartis | |
| Arcus Biosciences | anti-LAG-3 antibody, | Anti-LAG3 |
| | Arcus | |
| Ilgen | anti-LAG-3 antibody, | Anti-LAG3 |
| | Ilgen | |
| BJ Bioscience | BJ-007 | Anti-LAG3 |
| Xoma | ENUM-006 | Anti-LAG3 |
| Enumeral Biomedical | | |
| Microbio Group | SNA-03 | anti-lag3 |
| I-Mab Biopharma | TJ-A3 | Anti-LAG3 |

Several of these anti-LAG3 antibodies are described the U. S. patent documents. For example: US20170101472, U.S. Pat. Nos. 10,344,088, 6,143,273, 10,844,119, 9,908,936, 10,344,089, 9,505,839, US20140286935, U.S. Pat. Nos. 10,344,089, 10,266,591, 10,188,730, US20210095020.

In another aspect, the disclosure is directed to a multi variable domain immunoglobulin comprising the following an amino acid sequence having the following (Formula I):

[VH1]1-[L2]-[VH12]-[L1]-[VH3]-[HCCD1]

In this aspect, HCCD1 includes a first heavy chain constant domain of an antibody comprising a CH1 and a first Fc region (FC1). The antibody may be, for example an IgG antibody such as an anti-LAG antibody. VH3 includes a heavy chain variable domain of an anti-LAG3 antibody, and VH1 and VH2 represent one or the other of the IL2-R gamma VH and the IL2-R beta VH serially linked to the N-terminus of the VH3. And L1 and L2 are independently optional and, when present, may independently include an amino acid sequence of 2-20 amino acids.

As described above, IL2-R beta VH includes a framework region of a heavy chain variable region of a human antibody and three complimentary determining regions (CDRs) as follows:
CDR1 comprises an amino acid sequence selected from SEQ ID NO: 17, SEQ ID NO:18, SEQ ID NO: 19, SEQ ID NO: 37, SEQ ID NO: 38, or SEQ ID NO: 39,
CDR2 comprises an amino acid sequence selected from SEQ ID NO: 20, SEQ ID NO:21 or SEQ ID NO: 22, and
CDR3 comprises an amino acid sequence selected from SEQ ID NO: 23, SEQ ID NO: 40, or SEQ ID NO: 41;

Also, as described above, the IL2-R gamma VH comprises a framework region of a heavy chain variable region of a human antibody and three complimentary determining regions (CDRs) as follows,
CDR1 comprises an amino acid sequence selected from SEQ ID NO: 10, SEQ ID NO:11 or SEQ ID NO: 12,
CDR2 comprises an amino acid sequence selected from SEQ ID NO: 13, SEQ ID NO:14 or SEQ ID NO: 15, and
CDR3 comprises an amino acid sequence selected from SEQ ID NO: 16.

The IL2-R beta VH and the IL2-R gamma VH may be serially linked to the N-terminus of the VH3 in any order, and the linkage to the antibody or each other may optionally, and independently, include linker L1 and/or L2, which may be the same or different amino acid sequence of 2-20 amino acids. In example embodiments of Formula I, VH1 is the IL2-R gamma VH, and VH2 is the IL2-R beta VH. In another embodiment, VH2 is the IL2-R beta VH, and VH1 is the IL2-R gamma VH.

The IL2-R gamma VH1 may include the amino acid sequence of SEQ ID NO: 1. The IL-R beta VH may include one of SEQ ID NO: 2, SEQ ID NO: 35, SEQ ID NO: 36, or SEQ ID NO: 42. Each of these sequences include the regions of the VH that contain the amino acids for each CDR under any of the numbering systems.

As noted above, the HCCD of Formula 1 refers to a first heavy chain constant domain of an antibody and includes a CH1 region and a first Fc region (FC1). FC1 may include a wild type Fc (e.g. a human IgG Fc) or variants thereof. Accordingly, one aspect of the disclosure is directed to an Fc region (E.g., an IgG Fc) that has been modified to enhance dimerization and stability of a dimeric Fc, increase molecule stabilization generally, or reduce effector function. Modified Fc regions may be referred to generally as an “variant Fc polypeptide” or “Fc variant”. These include an Fc polypeptide derived from a parental Fc polypeptide. In one aspect of the disclosure, the Fc variant differs from the parental Fc polypeptide in that it includes stabilizing one or more stabilizing amino acid residues, e.g., due to the introduction of at least one Fc stabilizing mutation. In certain embodiments, the Fc variants of the invention comprise an Fc region (or Fc moiety) that is identical in sequence to that of a parental polypeptide but for the presence of one or more stabilizing Fc amino acids. In preferred embodiments, the Fc variant will have enhanced stability as compared to the parental Fc polypeptide and, optionally, equivalent or reduced effector function as compared to the parental Fc polypeptide.

In various embodiments of the disclosure, FC1 of Formula I may be one of SEQ ID NO: 44 (“HET 1 Fc”), SEQ ID NO: 45 (“HET 2 Fc”) or SEQ ID NO: 46 (“IgG Fc”). SEQ ID NO: 46 reflects a wild type IgG Fc as shown below:

IgG Fc (SEQ ID NO: 46)
(SEQ ID NO: 46)
DKTHTCPPCPAPELLGGPSVELFPPKPKDTLMISRTPEVTCVVVD
VSHEDPEVKENWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVESCSVMHEALHNHYTQKSLSLSP
GK

SEQ ID NO: 44 and SEQ ID NO: 45 reflect mutations to introduce residues have either positive and negative charges in order to stabilize a dimer formed between the sequences. In particular, SEQ ID NO: 44 includes mutations K370E, R409D, and K439E (numbering by Kabat) and SEQ ID NO: 45 includes mutations E356K/E357K/D399K (Kabat) as shown below:

HET 1 Fc (SEQ ID NO: 44)
(SEQ ID NO: 44)
DKTHTCPPCPAPEAAGGPSVELFPPKPKDTLMISRTPEVTCVVVD
VSHEDPEVKENWYVDGVEVHNAKTKPREEQYNSTYRVVSVLTVLH
QDWLNGKEYKCAVSNKALPAPIEKTISKAKGQPREPQVYTLPPSR
EEMTKNQVSLTCLVEGFYPSDIAVEWESNGQPENNYKTTPPVLDS
DGSFFLYSDLTVDKSRWQQGNVESCSVMHEALHNHYTQESLSLSP
GK
Het 2 Fc (SEQ ID NO: 45)
(SEQ ID NO: 45)
DKTHTCPPCPAPEAAGGPSVFLFPPKPKDTLMISRTPEVTCVVVD
VSHEDPEVKENWYVDGVEVHNAKTKPREEQYNSTYRVVSVLTVLH
QDWLNGKEYKCAVSNKALPAPIEKTISKAKGQPREPQVYTLPPSR
KKMTKNQVSLTCLVKGFYPSDIAVEWESNGQPENNYKTTPPVLKS
DGSFFLYSKLTVDKSRWQQGNVESCSVMHEALHNHYTQKSLSLSP
GK

In addition, a number of other approaches are known to address stabilization of a Fe heterodimer. For example: K392D/K409D and E356K/D399K (“DD-KK”); T366W (knob) and T366S/L368A/Y407V (hole); S267K/L368D/K370S with S267K/S364K/E357Q; S364H/T394F and Y349T/F405A (1F-TA); and S364H/F405A and Y349T/T394F (HA-TF). Moreover, any of the foregoing can further include a cysteine clamp to further stabilize the heterodimer, such as S354C with Y349C.

In aspects of the disclosure, FC1 of Formula 1 may include an Fc region has been modified to reduce or eliminate FcγR and/or C1q binding and effector function. In the aspects of the disclosure shown in SEQ ID NO: 44, SEQ ID NO: 45 and SEQ ID NOS: 111 to 114, all sequences reflect the following mutations to SEQ ID NO: 44 (IgG Fc): L234A, L235A, and K322A (numbering by Kabat), while SEQ ID NOS: 107 to 110 and SEQ ID NOS: 115 to 116 reflect L234A, L235A mutations but lack K322A.

In addition, a number of additional mutations or combinations of mutations are known to reduce or eliminate effector function. For example, N297A, N297G, N297Q, L234A/L235A, L234A/L235A/P329S, L234A/L235A/P329G, L234A/L235A/P331S, L235A/G237A/E318A, L234F/L235E/P331S, L234F/L235E/D265A, S228P/L235A, G236R/L328R, S298G/T299A, L234F/L235E/P331S, E233P/L234V/L235A/G236del/S267K, and V234A/G237A/P238S/H268A/V309L/A330S/P331S.

Accordingly, in aspects of the disclosure, FC1 of Formula 1 may be one of SEQ ID NO: 107 to SEQ ID NO: 116, each a variant of SEQ ID NO: 46 with mutations introduced to stabilize a dimer formed by each Het 1/Het 2 pair and/or to reduce or eliminate FcγR and/or C1q binding and effector function.

Het 1 Fc (variant 1; example 12; SEQ ID NO: 107)
(SEQ ID NO: 107)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRE
EMTKNQVSLTCLVEGFYPSDIAVEWESNGQPENNYKTTPPVLDSDGSFFLYSDLTVDKSRWQQGNVES
CSVMHEALHNHYTQESLSLSPGK
Het 2 Fc (variant 1; example 12; SEQ ID NO: 108)
(SEQ ID NO: 108)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRK
KMTKNQVSLTCLVKGFYPSDIAVEWESNGQPENNYKTTPPVLKSDGSFFLYSKLTVDKSRWQQGNVES
CSVMHEALHNHYTQKSLSLSPGK
Het 1 Fc (variant 2; example 12; SEQ ID NO: 109)
(SEQ ID NO: 109)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVCTLPPSRE
EMTKNQVSLWCLVKGFYPSDIAVEWESNGQPENNYKTTPPVLDSDGSFFLYSKLTVDKSRWQQGNVES
CSVMHEALHNHYTQKSLSLSPGK
Het 2 Fc (variant 2; example 12; SEQ ID NO: 110)
(SEQ ID NO: 110)
DKTHTCPPCPAPEAAGGPSVELFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPCRE
EMTKNQVSLSCAVKGFYPSDIAVEWESNGQPENNYKTTPPVLDSDGSFFLVSKLTVDKSRWQQGNVES
CSVMHEALHNHYTQKSLSLSPGK
Het 1 Fc (variant 3; example 12; SEQ ID NO: 111)
(SEQ ID NO: 111)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCAVSNKALPAPIEKTISKAKGQPREPQVCTLPPSRE
EMTKNQVSLWCLVKGFYPSDIAVEWESNGQPENNYKTTPPVLDSDGSFFLYSKLTVDKSRWQQGNVES
CSVMHEALHNHYTQKSLSLSPGK
Het 2 Fc (variant 3; example 12; SEQ ID NO: 112)
(SEQ ID NO: 112)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCAVSNKALPAPIEKTISKAKGQPREPQVYTLPPCRE
EMTKNQVSLSCAVKGFYPSDIAVEWESNGQPENNYKTTPPVLDSDGSFFLVSKLTVDKSRWQQGNVES
CSVMHEALHNHYTQKSLSLSPGK
Het 1 Fc (variant 4; example 12; SEQ ID NO: 113)
(SEQ ID NO: 113)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCAVSNKALPAPIEKTISKAKGQPREPQVCTLPPSRE
EMTKNQVSLWCLVKGFYPSDIAVEWESNGQPENNYKTTPPVLDSDGSFFLYSKLTVDKSRWQQGNVES
CSVMHEALHNHYTQKSLSLSPGK
Het 2 Fc (variant 4; example 12; SEQ ID NO: 114)
(SEQ ID NO: 114)
DKTHTCPPCPAPEAAGGPSVFLFPPKPKDTLMISRTPEVTCVVVDVSHEDPEVKENWYVDGVEVHNAK
TKPREEQYNSTYRVVSVLTVLHQDWLNGKEYKCAVSNKALPAPIEKTISKAKGQPREPQVYTLPPCRE
EMTKNQVSLSCAVKGFYPSDIAVEWESNGQPENNYKTTPPVLDSDGSFFLVSKLTVDKSRWQQGNVES
CSVMHEALHNHYTQKSLSLSPGK
Het 1 Fc (variant 5; example 12; SEQ ID NO: 115)
(SEQ ID NO: 115)
DKTHTCPPCPAPELLGGPSVFCFPPKPKDTLMISRTPEVTCVVVDVSHEDPEVKENWYVDGVEVHNAK
TKPREEQYGSTYRVVSVLTVLHQDWLNGKEYKCKVSNKALPAPIECTISKAKGQPREPQVYTLPPSRE
EMTKNQVSLTCLVEGFYPSDIAVEWESNGQPENNYKTTPPVLDSDGSFFLYSDLTVDKSRWQQGNVES
CSVMHEALHNHYTQESLSLSPGK
Het 2 Fc (variant 5; example 12; SEQ ID NO: 116)
(SEQ ID NO: 116)
DKTHTCPPCPAPELLGGPSVFCFPPKPKDTLMISRTPEVTCVVVDVSHEDPEVKENWYVDGVEVHNAK
TKPREEQYGSTYRVVSVLTVLHQDWLNGKEYKCKVSNKALPAPIECTISKAKGQPREPQVYTLPPSRK
KMTKNQVSLTCLVKGFYPSDIAVEWESNGQPENNYKTTPPVLKSDGSFFLYSKLTVDKSRWQQGNVES
CSVMHEALHNHYTQKSLSLSPGK

In various aspects of the disclosure, FC1 of Formula I may be SEQ ID NO: 44, 45, 46, 107, 108, 109, 110, 111, 112, 113, 114, 115 or 116, or may include other mutations as noted herein and known in the art.

Also, the HCCD of Formula 1 includes an CH1 region of an antibody, such as an IgG HC1. An example of a suitable amino acid sequence for CH1 includes the following:

(SEQ ID NO: 47)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSC

Accordingly, examples of amino acid sequences of complete HCCD, including an FC1 and CH1, are the following (designated herein “IgG Het1,” “IgG Het2” and “IgG HC”, and variants thereof):

IgG Het1
(SEQ ID NO: 4)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKENWYVDGVEVHNAKTKPREEQYNSTYRVVSVLTVLHQDWLNGKEYKC
AVSNKALPAPIEKTISKAKGQPREPQVYTLPPSREEMTKNQVSLTCLVEGFYPSDIAVEWESNGQPEN
NYKTTPPVLDSDGSFFLYSDLTVDKSRWQQGNVFSCSVMHEALHNHYTQESLSLSPGK
IgG HET2
(SEQ ID NO: 5)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKENWYVDGVEVHNAKTKPREEQYNSTYRVVSVLTVLHQDWLNGKEYKC
AVSNKALPAPIEKTISKAKGQPREPQVYTLPPSRKKMTKNQVSLTCLVKGFYPSDIAVEWESNGQPEN
NYKTTPPVLKSDGSFFLYSKLTVDKSRWQQGNVFSCSVMHEALHNHYTQKSLSLSPGK
IgG HC
(SEQ ID NO: 43)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLMI
SRTPEVTCVVVDVSHEDPEVKENWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK.
IgG HET1 (variant 1; Example 12)
(SEQ ID NO: 96)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVELFPPKPKDTLMI
SRTPEVTCVVVDVSHEDPEVKENWYVDGVEVHNAKTKPREEQYNSTYRVVSVLTVLHQDWLNGKEYKC
KVSNKALPAPIEKTISKAKGQPREPQVYTLPPSREEMTKNQVSLTCLVEGFYPSDIAVEWESNGQPEN
NYKTTPPVLDSDGSFFLYSDLTVDKSRWQQGNVFSCSVMHEALHNHYTQESLSLSPGK
IgG HET 2 (variant 1; Example 12)
(SEQ ID NO: 97)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKENWYVDGVEVHNAKTKPREEQYNSTYRVVSVLTVLHQDWLNGKEYKC
KVSNKALPAPIEKTISKAKGQPREPQVYTLPPSRKKMTKNQVSLTCLVKGFYPSDIAVEWESNGQPEN
NYKTTPPVLKSDGSFFLYSKLTVDKSRWQQGNVFSCSVMHEALHNHYTQKSLSLSPGK
IgG HET1 (variant 2; Example 12)
(SEQ ID NO: 98)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVELFPPKPKDTLMI
SRTPEVTCVVVDVSHEDPEVKENWYVDGVEVHNAKTKPREEQYNSTYRVVSVLTVLHQDWLNGKEYKC
KVSNKALPAPIEKTISKAKGQPREPQVCTLPPSREEMTKNQVSLWCLVKGFYPSDIAVEWESNGQPEN
NYKTTPPVLDSDGSFFLYSKLTVDKSRWQQGNVFSCSVMHEALHNHYTQKSLSLSPGK
IgG HET2 (variant 2; Example 12)
(SEQ ID NO: 99)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVELFPPKPKDTLMI
SRTPEVTCVVVDVSHEDPEVKENWYVDGVEVHNAKTKPREEQYNSTYRVVSVLTVLHQDWLNGKEYKC
KVSNKALPAPIEKTISKAKGQPREPQVYTLPPCREEMTKNQVSLSCAVKGFYPSDIAVEWESNGQPEN
NYKTTPPVLDSDGSFFLVSKLTVDKSRWQQGNVFSCSVMHEALHNHYTQKSLSLSPGK
IgG HET1 (variant 3; Example 12)
(SEQ ID NO: 100)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVELFPPKPKDTLMI
SRTPEVTCVVVDVSHEDPEVKENWYVDGVEVHNAKTKPREEQYNSTYRVVSVLTVLHQDWLNGKEYKC
AVSNKALPAPIEKTISKAKGQPREPQVCTLPPSREEMTKNQVSLWCLVKGFYPSDIAVEWESNGQPEN
NYKTTPPVLDSDGSFFLYSKLTVDKSRWQQGNVFSCSVMHEALHNHYTQKSLSLSPGK
IgG HET2 (variant 3; Example 12)
(SEQ ID NO: 101)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKENWYVDGVEVHNAKTKPREEQYNSTYRVVSVLTVLHQDWLNGKEYKC
AVSNKALPAPIEKTISKAKGQPREPQVYTLPPCREEMTKNQVSLSCAVKGFYPSDIAVEWESNGQPEN
NYKTTPPVLDSDGSFFLVSKLTVDKSRWQQGNVFSCSVMHEALHNHYTQKSLSLSPGK
IgG HET1 (variant 4; Example 12)
(SEQ ID NO: 102)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKENWYVDGVEVHNAKTKPREEQYNSTYRVVSVLTVLHQDWLNGKEYKC
AVSNKALPAPIEKTISKAKGQPREPQVCTLPPSREEMTKNQVSLWCLVKGFYPSDIAVEWESNGQPEN
NYKTTPPVLDSDGSFFLYSKLTVDKSRWQQGNVFSCSVMHEALHNHYTQKSLSLSPGK
IgG HET2 (variant 4; Example 12)
(SEQ ID NO: 103)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKENWYVDGVEVHNAKTKPREEQYNSTYRVVSVLTVLHQDWLNGKEYKC
AVSNKALPAPIEKTISKAKGQPREPQVYTLPPCREEMTKNQVSLSCAVKGFYPSDIAVEWESNGQPEN
NYKTTPPVLDSDGSFFLVSKLTVDKSRWQQGNVFSCSVMHEALHNHYTQKSLSLSPGK
IgG HET1 (variant 5; Example 12)
(SEQ ID NO: 104)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFCFPPKPKDTLMI
SRTPEVTCVVVDVSHEDPEVKENWYVDGVEVHNAKTKPREEQYGSTYRVVSVLTVLHQDWLNGKEYKC
KVSNKALPAPIECTISKAKGQPREPQVYTLPPSREEMTKNQVSLTCLVEGFYPSDIAVEWESNGQPEN
NYKTTPPVLDSDGSFFLYSDLTVDKSRWQQGNVFSCSVMHEALHNHYTQESLSLSPGK
IgG HET2 (variant 5; Example 12)
(SEQ ID NO: 105)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFCFPPKPKDTLMI
SRTPEVTCVVVDVSHEDPEVKENWYVDGVEVHNAKTKPREEQYGSTYRVVSVLTVLHQDWLNGKEYKC
KVSNKALPAPIECTISKAKGQPREPQVYTLPPSRKKMTKNQVSLTCLVKGFYPSDIAVEWESNGQPEN
NYKTTPPVLKSDGSFFLYSKLTVDKSRWQQGNVFSCSVMHEALHNHYTQKSLSLSPGK

IgG Het1 and IgG Het2 and variants thereof (SEQ ID NO: 4, SEQ ID NO: 5, and SEQ ID NOS: 96 to 105) include the mutations in the Fc regions as described above for SEQ ID NO: 44, SEQ ID NO: 45, and SEQ ID NOS: 107 to 116. IgG HC (SEQ ID NO: 43) does not include the mutations and represents a wild type IgG HC1.

As noted above, the VH3 of Formula I includes heavy chain variable domain of an anti-LAG3 antibody, which would be linked to the N-terminus of CH1. An example of a heavy chain variable domain of anti-LAG 3 antibody includes the following CDRs:

| LAG3 | Numbering | | SEQ ID |
|---|---|---|---|
| CDR | System | Sequence | NO: |
| 1 | M | GFTFSSYGMH | 24 |
| 1 | K | SYGMH | 25 |
| 1 | C | GFTESSY | 26 |
| 2 | M | VIWYDGSNKY | 27 |
| 2 | K | VIWYDGSNKYYADSVK | 28 |
| 2 | C | WYD | 29 |
| 3 | M, K, C | EWAVASWDYGMDV | 30 |

All of these CDRs are contained in a framework region of the anti-LAG3 antibody that provides the complete anti-LAG3 VH, which may be, for example SEQ ID NO: 3 as follows:

(SEQ ID NO: 3)
QVQLVESGGGVVQPGRSLRLSCAASGFTFSSYGMHWVRQAPGK
GLEWVAVIWYDGSNKYYADSVKGRETISRDNSKNTLYLQMNSL
RAEDTAVYYCAREWAVASWDYGMDVWGQGTTVTVSS

Underlined portions of the sequence reflect the regions of the VH that contain the amino acids for each CDR under any of the numbering systems.

### Linkers

The multi immunoglobulin variable domains and dimeric molecules of the disclosure may include one or more linkers between the variable heavy chain domains, the LAG3 antibody, or an Fc region. Linkers are optional, and may be of any length, for example 2-20 amino acids, more particularly, 2, 3, 4, 5, 6, 7, 8, 9, 10, 11, 12, 13, 14, 15, 16, 17, 18, 19 or 20 amino acids.

Examples of useful linkers include the following:

PP
GG
(SEQ ID NO: 52)
PPGG
(SEQ ID NO: 53)
PPGGQGQGGQGGGQGGGPP
(SEQ ID NO: 54)
GGQGQGGQGGG
(SEQ ID NO: 55)
GGSGS
(SEQ ID NO: 56)
GGGSGS
(SEQ ID NO: 57)
GGGGS
(SEQ ID NO: 58)
GGGGSGGGS
(SEQ ID NO: 59)
GGGGSGGGGSGGGGS
(SEQ ID NO: 60)
GGGGSGGGGSAS
(SEQ ID NO: 61)
GGQGQGGQGGGQGGG
(SEQ ID NO: 62)
GGQG
(SEQ ID NO: 63)
GGQGQGG
(SEQ ID NO: 64)
PPGGGGS
(SEQ ID NO: 65)
PPGGGGSGGGS
(SEQ ID NO: 66)
PPGGGGSGGGGSGGGGS
(SEQ ID NO: 67)
PPGGGGSGGGGSAS
(SEQ ID NO: 68)
PPGGQGQGGQGGGQGGG
(SEQ ID NO: 69)
PPGGQG
(SEQ ID NO: 70)
PPGGQGQGG
(SEQ ID NO: 71)
PPGGQGQGGQGGG
(SEQ ID NO: 72)
GSTSGSGKPGSGEGSTKG
(SEQ ID NO: 73)
IRPRAIGGSKPRVA
(SEQ ID NO: 74)
GKGGS
(SEQ ID NO: 75)
GKGGSGKGGSG
(SEQ ID NO: 76)
GKGGSGKGGSGKGGS
(SEQ ID NO: 77)
STAGDTHLGGEDFD
(SEQ ID NO: 78)
PPGSTSGSGKPGSGEGSTKG
(SEQ ID NO: 79)
PPIRPRAIGGSKPRVA
(SEQ ID NO: 80)
PPGKGGS
(SEQ ID NO: 81)
PPGKGGSGKGGSG
(SEQ ID NO: 82)
PPGKGGSGKGGSGKGGS
(SEQ ID NO: 83)
PPSTAGDTHLGGEDFD
(SEQ ID NO: 84)
SDKTHT
(SEQ ID NO: 85)
PPSDKTHT
(SEQ ID NO: 106)
PPGGSGGGGSGGGGSGG

In particular aspects of the disclosure, the linker for linking a VH1 or a VH2 to the VH3 (e.g., L2 of Formula 1) is selected from GG, PP, PPGG (SEQ ID NO: 52), GGGSGS (SEQ ID NO: 56), GGQGQGGQGGGQGGG (SEQ ID NO: 61), or PPGGQGQGGQGGGQGGG (SEQ ID NO: 68). In addition, in embodiments, the linker for linking VH1 to VH2 (e.g, L1 of Formula 1) may be one of GG, PP, PPGG (SEQ ID NO: 52), GGGSGS (SEQ ID NO: 56), GGQGQGGQGGGQGGG (SEQ ID NO: 61), or PPGGQGQGGQGGGQGGG (SEQ ID NO: 68).

Examples of complete single chain multivariable domain immunoglobulins.

In various examples, Formula 1 provides a multi domain immunoglobulin including the following sequences:

VH1 = SEQ ID NO: 1,
L1 =
(SEQ ID NO: 52)
GG, PP, PPGG,
(SEQ ID NO: 56)
GGGSGS;
(SEQ ID NO: 61)
GGQGQGGQGGGQGGG,
or
(SEQ ID NO: 68)
PPGGQGQGGQGGGQGGG
VH2 = SEQ ID NO: 2, SEQ ID NO: 35
SEQ ID NO: 36, or SEQ ID NO: 42,
L2 =
(SEQ ID NO: 52)
GG, PP, PPGG,
(SEQ ID NO: 56)
GGGSGS;
(SEQ ID NO: 61)
GGQGQGGQGGGQGGG,
or
(SEQ ID NO: 68)
PPGGQGQGGQGGGQGGG.
VH3 = SEQ ID NO: 3,
and
HCCD = SEQ ID NO: 4, SEQ ID NO: 5,
SEQ ID NO: 43, SEQ ID NO: 96,
SEQ ID NO: 97, SEQ ID NO: 98,
SEQ ID NO: 99, SEQ ID NO: 100,
SEQ ID NO: 101, SEQ ID NO: 102,
SEQ ID NO: 103, SEQ ID NO: 104,
or
SEQ ID NO: 105.

In one example, VH1 is SEQ ID NO:1, L1 is PPGG (SEQ ID NO: 52), VH2 is SEQ ID NO: 2, L2 is PPGGQGQGGQGGGQGGG (SEQ ID NO: 68), VH3 is SEQ ID NO:3 and HCCD is SEQ ID NO: 4 (IgG Het1), which in series provides the following amino acid sequence:

(SEQ ID NO: 48)
EVQLVESGGGVVRPGGSLRLSCCASGFTEDDYGMSWVRRAPGKGL
EWVAGINWNGGSTGYADSVKGRFTISRDNEKNCLYLQMNSLRAED
TALYHCASATSGTAFDIWGQGTMVTVSSPPGGEVQLVQSGAEVKK
PGACVKISCKVSGYTFTRYYMHWVQRAPGKGLEWIGLVDPEDGET
IYAEKFQGRVTITADTSTDTAYMELCSLRSEDTAVYYCATDFVKL
DFSLSFQHWGQGTLVTVSSPPGGQGQGGQGGGQGGGQVQLVESGG
GVVQPGRSLRLSCAASGFTFSSYGMHWVRQAPGKGLEWVAVIWYD
GSNKYYADSVKGRFTISRDNSKNTLYLQMNSLRAEDTAVYYCARE
WAVASWDYGMDVWGQGTTVTVSSASTKGPSVFPLAPSSKSTSGGT
AALGCLVKDYFPEPVTVSWNSGALTSGVHTFPAVLQSSGLYSLSS
VVTVPSSSLGTQTYICNVNHKPSNTKVDKKVEPKSCDKTHTCPPC
PAPEAAGGPSVELFPPKPKDTLMISRTPEVTCVVVDVSHEDPEVK
ENWYVDGVEVHNAKTKPREEQYNSTYRVVSVLTVLHQDWLNGKEY
KCAVSNKALPAPIEKTISKAKGQPREPQVYTLPPSREEMTKNQVS
LTCLVEGFYPSDIAVEWESNGQPENNYKTTPPVLDSDGSFFLYSD
LTVDKSRWQQGNVFSCSVMHEALHNHYTQESLSLSPGK.

In another example, VH1 is SEQ ID NO:1, L1 is PPGG (SEQ ID NO: 52), VH2 is SEQ ID NO: 2, L2 is PPGGQGQGGQGGGQGGG (SEQ ID NO: 68), VH3 is SEQ ID NO:3 and HCCD is SEQ ID NO: 5 (IgG Het2), which in series provides the following amino acid sequence:

(SEQ ID NO: 88)
EVQLVESGGGVVRPGGSLRLSCCASGFTEDDYGMSWVRRAPGKGL
EWVAGINWNGGSTGYADSVKGRFTISRDNEKNCLYLQMNSLRAED
TALYHCASATSGTAFDIWGQGTMVTVSSPPGGEVQLVQSGAEVKK
PGACVKISCKVSGYTFTRYYMHWVQRAPGKGLEWIGLVDPEDGET
IYAEKFQGRVTITADTSTDTAYMELCSLRSEDTAVYYCATDFVKL
DFSLSFQHWGQGTLVTVSSPPGGQGQGGQGGGQGGGQVQLVESGG
GVVQPGRSLRLSCAASGFTFSSYGMHWVRQAPGKGLEWVAVIWYD
GSNKYYADSVKGRFTISRDNSKNTLYLQMNSLRAEDTAVYYCARE
WAVASWDYGMDVWGQGTTVTVSSASTKGPSVFPLAPSSKSTSGGT
AALGCLVKDYFPEPVTVSWNSGALTSGVHTFPAVLQSSGLYSLSS
VVTVPSSSLGTQTYICNVNHKPSNTKVDKKVEPKSCDKTHTCPPC
PAPEAAGGPSVELFPPKPKDTLMISRTPEVTCVVVDVSHEDPEVK
ENWYVDGVEVHNAKTKPREEQYNSTYRVVSVLTVLHQDWLNGKEY
KCAVSNKALPAPIEKTISKAKGQPREPQVYTLPPSRKKMTKNQVS
LTCLVKGFYPSDIAVEWESNGQPENNYKTTPPVLKSDGSFFLYSK
LTVDKSRWQQGNVFSCSVMHEALHNHYTQKSLSLSPGK

In another example, VH1 is SEQ ID NO:1, L1 is PPGG (SEQ ID NO: 52), VH2 is SEQ ID NO: 2, L2 is PPGGQGQGGQGGGQGGG (SEQ ID NO: 68), VH3 is SEQ ID NO:3 and HCCD is SEQ ID NO: 96, which in series provides the following amino acid sequence:

(SEQ ID NO: 86)
EVQLVESGGGVVRPGGSLRLSCCASGFTEDDYGMSWVRRAPGKGL
EWVAGINWNGGSTGYADSVKGRFTISRDNEKNCLYLQMNSLRAED
TALYHCASATSGTAFDIWGQGTMVTVSSPPGGEVQLVQSGAEVKK
PGACVKISCKVSGYTFTRYYMHWVQRAPGKGLEWIGLVDPEDGET
IYAEKFQGRVTITADTSTDTAYMELCSLRSEDTAVYYCATDFVKL
DESLSFQHWGQGTLVTVSSPPGGQGQGGQGGGQGGGQVQLVESGG
GVVQPGRSLRLSCAASGFTFSSYGMHWVRQAPGKGLEWVAVIWYD
GSNKYYADSVKGRFTISRDNSKNTLYLQMNSLRAEDTAVYYCARE
WAVASWDYGMDVWGQGTTVTVSSASTKGPSVFPLAPSSKSTSGGT
AALGCLVKDYFPEPVTVSWNSGALTSGVHTFPAVLQSSGLYSLSS
VVTVPSSSLGTQTYICNVNHKPSNTKVDKKVEPKSCDKTHTCPPC
PAPEAAGGPSVELFPPKPKDTLMISRTPEVTCVVVDVSHEDPEVK
ENWYVDGVEVHNAKTKPREEQYNSTYRVVSVLTVLHQDWLNGKEY
KCKVSNKALPAPIEKTISKAKGQPREPQVYTLPPSREEMTKNQVS
LTCLVEGFYPSDIAVEWESNGQPENNYKTTPPVLDSDGSFFLYSD
LTVDKSRWQQGNVFSCSVMHEALHNHYTQESLSLSPGK

In another example, VH1 is SEQ ID NO:1, L1 is PPGG (SEQ ID NO: 52), VH2 is SEQ ID NO: 2, L2 is PPGGQGQGGQGGGQGGG (SEQ ID NO: 68), VH3 is SEQ ID NO:3 and HCCD is SEQ ID NO: 98, which in series provides the following amino acid sequence:

(SEQ ID NO: 89)
EVQLVESGGGVVRPGGSLRLSCCASGFTEDDYGMSWVRRAPGKGL
EWVAGINWNGGSTGYADSVKGRFTISRDNEKNCLYLQMNSLRAED
TALYHCASATSGTAFDIWGQGTMVTVSSPPGGEVQLVQSGAEVKK
PGACVKISCKVSGYTFTRYYMHWVQRAPGKGLEWIGLVDPEDGET
IYAEKFQGRVTITADTSTDTAYMELCSLRSEDTAVYYCATDFVKL
DESLSFQHWGQGTLVTVSSPPGGSGGGGSGGGGSGGQVQLVESGG
GVVQPGRSLRLSCAASGFTESSYGMHWVRQAPGKGLEWVAVIWYD
GSNKYYADSVKGRFTISRDNSKNTLYLQMNSLRAEDTAVYYCARE
WAVASWDYGMDVWGQGTTVTVSSASTKGPSVFPLAPSSKSTSGGT
AALGCLVKDYFPEPVTVSWNSGALTSGVHTFPAVLQSSGLYSLSS
VVTVPSSSLGTQTYICNVNHKPSNTKVDKKVEPKSCDKTHTCPPC
PAPEAAGGPSVELFPPKPKDTLMISRTPEVTCVVVDVSHEDPEVK
ENWYVDGVEVHNAKTKPREEQYNSTYRVVSVLTVLHQDWLNGKEY
KCKVSNKALPAPIEKTISKAKGQPREPQVCTLPPSREEMTKNQVS
LWCLVKGFYPSDIAVEWESNGQPENNYKTTPPVLDSDGSFFLYSK
LTVDKSRWQQGNVFSCSVMHEALHNHYTQKSLSLSPGK

### Multi Variable Domain Immunoglobulins Including a Light Chain

Any of the foregoing multi variable domain immunoglobulins may further include a light chain of an anit-LAG3 antibody (VL1) of a complete anti-LAG3 light chain (LC1). A VL1 may include framework region of a light chain variable region of an anti-LAG3 antibody and three complimentary determining regions (CDRs) according to the following:

| | Numbering | | SEQ ID |
|---|---|---|---|
| CDR | System | Sequence | NO: |
| 1 | M, K, C | RASQSVSSSYLA | 31 |
| 2 | M, K, C | GASSRAT | 32 |
| 3 | M, K, C | QQYGSSPFT | 33 |

All of these CDRs are contained in a framework region of a light chain variable region of an anti-LAG3 antibody that provides the complete the anti-LAG3 VL, which may be, for example SEQ ID NO: 6 as follows:

(SEQ ID NO: 6)
EIVLTQSPGTLSLSPGERATLSCRASQSVSSSYLAWYQQ
KPGQAPRLLIYGASSRATGIPDRFSGSGSGTDFTLTISR
LEPEDFAVYYCQQYGSSPFTFGPGTKVDIK

Underlined portions of the sequence reflect the regions of the VL that contain the amino acids for each CDR under any of the numbering systems.

In addition, the anti-LAG3 VL may include an affinity matured variant in CDR3 and may include the following CDRs:

| | Numbering | | SEQ ID |
|---|---|---|---|
| CDR | System | Sequence | NO: |
| 1 | M, K, C | RASQSVSSSYLA | 31 |
| 2 | M, K, C | GASSRAT | 32 |
| 3 | M, K, C | QQYGESPFT | 34 |

All of these CDRs are contained in a framework region of a light chain variable region of an anti-LAG3 antibody that provides the complete the anti-LAG3 VL, which may be, for example SEQ ID NO: 8 as follows:

(SEQ ID NO: 8)
EIVLTQSPGTLSLSPGERATLSCRASQSVSSSYLAWYQQ
KPGQAPRLLIYGASSRATGIPDRESGSGSGTDFTLTISR
LEPEDFAVYYCQQYGESPFTFGPGTKVDIK

Underlined portions of the sequence reflect the regions of the VL that contain the amino acids for each CDR under any of the numbering systems.

Examples of complete anti-LAG3 light chains include the following amino acid sequences:

(SEQ ID NO: 7)
EIVLTQSPGTLSLSPGERATLSCRASQSVSSSYLAWYQQKPGQAP
RLLIYGASSRATGIPDRESGSGSGTDFTLTISRLEPEDFAVYYCQ
QYGSSPFTFGPGTKVDIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSENRGEC
(SEQ ID NO: 9)
EIVLTQSPGTLSLSPGERATLSCRASQSVSSSYLAWYQQKPGQAP
RLLIYGASSRATGIPDRESGSGSGTDFTLTISRLEPEDFAVYYCQ
QYGESPFTFGPGTKVDIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSENRGEC

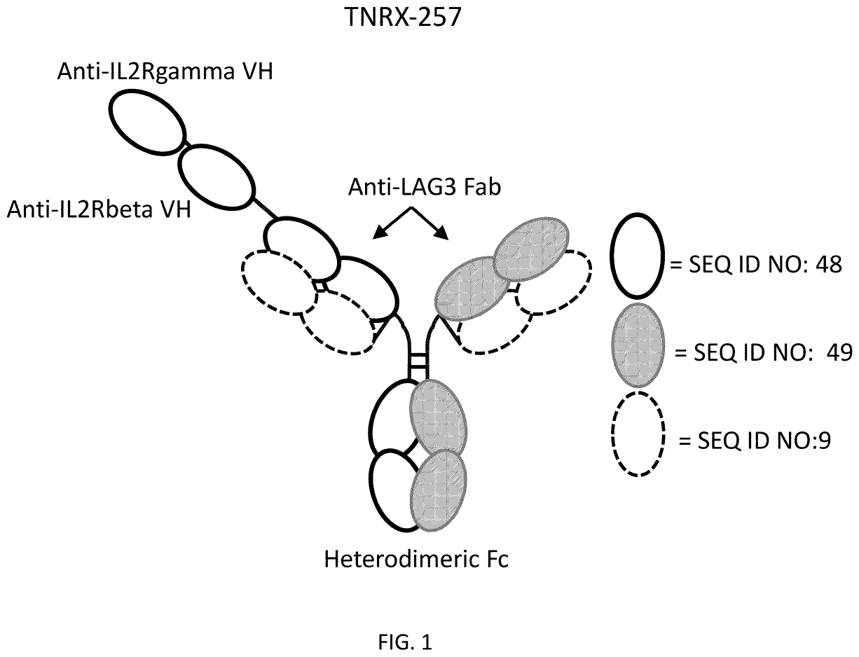

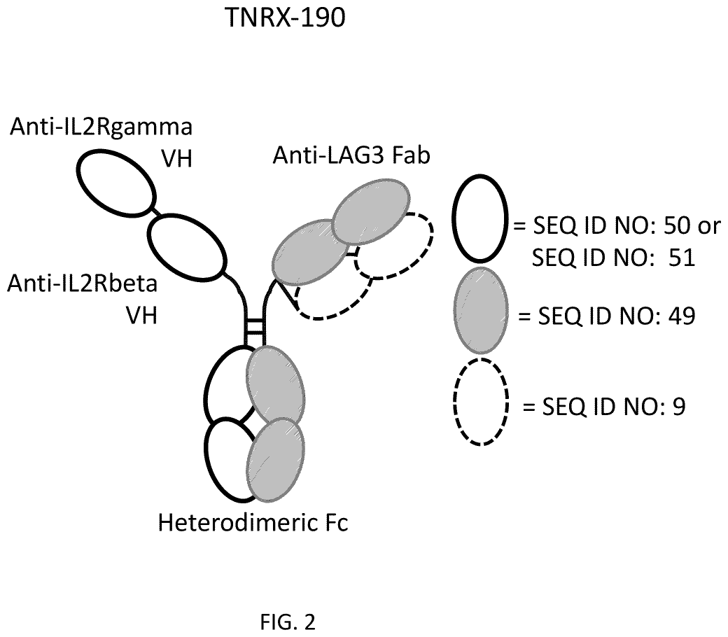

The VL or LC of the disclosure may associate with the anti-LAG 3 HC and VH's to form partial or complete anti-LAG3 antibodies as shown in FIGS. 1 and 2.

### Heterodimer Molecules

In another aspect, the disclosure is directed to a heterodimer molecule including the single chain multi variable domain immunoglobulin of described above (e.g., amino acid sequence of Formula 1) and a second molecule that dimerizes with the immunoglobulin described above. For example, the second molecule include a second HCCD (“HCCD2”) that dimerizes with the sequence of Formula 1. HCCD2 includes an FC2 and an CH1, wherein the FC1 of HCCD1 and the FC2 of HCCD2 associate by charge or otherwise to form a heterodimeric molecule (or complex). As noted above, FC1 and FC2 may include a number of mutations that promote the heterodimerization of the Fcs and corresponding molecule. CH2 may be the same as CH1.

Examples according to the disclosure include an HCCD1 according to SEQ ID NO: 4 (IgG Het1) and an HCCD2 according to SEQ ID NO: 5 (IgG Het2) that dimerize because of their mutations. In an alternative embodiment of the disclosure HCCD1 may include SEQ ID NO: 5 and HCCD2 may include SEQ ID NO: 4.

In each of these embodiments the heterodimeric molecule may include a LAG3 heavy chain variable region (VH3) attached one of both of HCs (HC1 and/or HC2) of the HCCDs, and may further include a an IL2-R gamma VH and/or an IL2-R beta VH linked singly or serially linked to the N-terminus of one or both VH3s of the heterodimeric molecule (with, optionally, the linkers described herein).

Embodiments of the heterodimeric molecule include the VLs and CL of an anti-LAG3 antibody associate with the CH1 and VH3 to complete the anti-LAG3 antibody. One example of a heterodimeric molecule of the disclosure is represented in FIG. 1, which shows a complete anti-LAG3 antibody having a IL2-R beta VH linked to the N-terminus of one of the VHs of the antibody and the IL2-R gamma VH linked to the N terminus of the IL-2R beta VH.

### Example Heterodimeric Molecule

An example heterodimeric molecule of the disclosure is shown in FIG. 2, which shows an IgG Fc heterodimer. One of the Fcs is linked to an anti-LAG3 Fab, including an CH1, an anti-LAG3 VH, and a LC including a light chain constant region and an anti-LAG3 light chain variable region. The other Fc is linked to an anti-IL2-R beta VH of the disclosure, which is linked to an anti-IL2-R gamma VH of the disclosure.

An embodiment of the heterodimeric molecule of the disclosure includes the following segments:
(a) a first heavy chain constant domain (HCCD1) of an IgG antibody comprising a CH1 and a first Fc region (FC1);
(b) an anti-LAG3 VH (VH3) linked to the N-terminus of the CH1;
(c) a LC including a light chain constant region and an anti-LAG3 light chain variable region (VL1);
(c) a second Fc region (FC2) that heterodimerizes with FC1;
(d) a single immunoglobulin variable domain (VH2) that binds to IL-2R beta that is linked the N-terminus of the FC2; and
(e) a single immunoglobulin variable domain (VH1) that binds to IL-2R gamma linked to the N-terminus of VH1.

In embodiments of the foregoing, the first HCC1 may include either (a) SEQ ID NO: 4 (IgG HET1), which includes an IgG Fc1 (SEQ ID NO: 44) and a CH1 (e.g., SEQ ID NO: 47), or (b) SEQ ID NO:5 (IgG HET2), which includes an IgG Fc2 (SEQ ID NO: 45) and a CH1 (e.g., SEQ ID NO: 47).

VH3 includes a framework region of a heavy chain variable region of anti-LAG3 antibody and three complimentary determining regions (CDRs). For example,
CDR1 includes an amino acid sequence selected from SEQ ID NO: 24, SEQ ID NO: 25 or SEQ ID NO: 26,
CDR2 includes an amino acid sequence selected from SEQ ID NO: 27, SEQ ID NO: 28 or SEQ ID NO: 29, and
CDR3 includes an amino acid sequence selected from SEQ ID NO: 30,

An example of VH3 is SEQ ID NO: 3.

The anti-LAG 3 light chain variable region of LC includes a light chain variable region of an anti-LAG3 antibody and three complimentary determining regions (CDRs), which may be one of SEQ ID NO: 6 or SEQ ID NO: 8. The complete LC may include SEQ ID NO: 7 or 9.

FC2 includes either (a) SEQ ID NO: 44 when HCCD1 comprises SEQ ID NO: 4, or (b) SEQ ID NO: 45 when HCCD1 comprises SEQ ID NO: 5.

VH1 includes a single immunoglobulin variable domain that binds to IL-2R gamma, comprising a framework region of a heavy chain variable region of a human antibody and three complimentary determining regions (CDRs). For example,
CDR1 includes an amino acid sequence selected from SEQ ID NO: 10, SEQ ID NO:11 or SEQ ID NO: 12,
CDR2 includes an amino acid sequence selected from SEQ ID NO: 13, SEQ ID NO:14 or SEQ ID NO: 15, and
CDR3 includes an amino acid sequence selected from SEQ ID NO: 16,

An example of VH1 is SEQ ID NO: 1.

VH2 includes single immunoglobulin variable domain that binds to IL-2R beta comprising a framework region of a heavy chain variable region of a human antibody and three complimentary determining regions (CDRs). For example,
CDR1 includes an amino acid sequence selected from SEQ ID NO: 17, SEQ ID NO:18, SEQ ID NO: 19, SEQ ID NO: 37, SEQ ID NO: 38, or SEQ ID NO: 39,
CDR2 includes an amino acid sequence selected from SEQ ID NO: 20, SEQ ID NO:21 or SEQ ID NO: 22, and
CDR3 includes an amino acid sequence selected from SEQ ID NO: 23, SEQ ID NO: 40, or SEQ ID NO: 41, and

Examples of VH2 include SEQ ID NO: 2, SEQ ID NO: 35, SEQ ID NO: 36, and SEQ ID NO: 42.

In the heterodimeric molecule of the disclosure the VH1 or VH2 may optionally be linked to the FC2 through an amino acid linker, for example an amino acid linker comprising 2-20 amino acids. Examples of suitable linkers include GG, PP, PPGG, (SEQ ID NO: 52), GGGSGS (SEQ ID NO: 56), GGQGQGGQGGGQGGG (SEQ ID NO: 61), or PPGGQGQGGQGGGQGGG (SEQ ID NO: 68),

In addition, when VH1 may optionally be linked to the VH2 through an amino acid linker, for example an amino acid linker comprising 2-20 amino acids. Examples of suitable linkers includes GG, PP, PPGG, (SEQ ID NO: 52), GGGSGS (SEQ ID NO: 56); GGQGQGGQGGGQGGG (SEQ ID NO: 61), or PPGGQGQGGQGGGQGGG (SEQ ID NO: 68).

### Pharmaceutical Formulations

In some embodiments of the disclosure, the single immunoglobulin variable domains or the heterodimeric molecules of the disclosure herein may be formulated with a pharmaceutically acceptable carrier, excipient, or stabilizer, as pharmaceutical compositions.

In certain embodiments, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances, which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

### Pharmaceutical Combinations

The single immunoglobulin variable domains or the heterodimeric molecules may be administered to a subject alone, or in combination (serially or sequentially) with an anti-PD1 antibody or an anti-PDL1 antibody that is also formulated for administration to a patient. A number of anti-PD1 antibodies and anti-PDL1 antibodies are known as show in Tables 2 and 3.

TABLE 2

| Company | Generic Drug Name | Target |
|---|---|---|
| Jiangsy | camrelizumab | PD1 |
| Regeneron | cemiplimab | PD1 |
| GSK/Anaptsys | dostarlimab | PD1 |
| BMS | nivolumab | PD1 |
| Merck | pembrolizumab | PD1 |
| Akeso | penpulimab | PD1 |
| Biocad | prolgolimab | PD1 |
| Shanghai Fosun | serplulimab | PD1 |
| Innovent | sintilimab | PD1 |
| Beigene | tislelizumab | PD1 |
| Shanghai Junshi | toripalimab | PD1 |
| Gilead/Hervin Gloria | zimberelimab | PD1 |
| Taizhou | pucotenlimab | PD1 |
| Agenus | balstilimab | PD1 |
| Genor | genolimzumab | PD1 |
| Macrogenics/Incyte | retifanlimab | PD1 |
| JnJ | cetrelimab | PD1 |
| Cstone | nofazinlimab | PD1 |
| Qiu | QL-1604 | PD1 |
| Pfizer | sasanlimab | PD1 |
| SinoceIltech | SCT-I10A | PD1 |
| CSPC | SYSA-1802 | PD1 |
| Amgen | AMG-404 | PD1 |
| Abbvie | budigalimab | PD1 |
| Boehringer Ingelheim | ezabenlimab | PD1 |
| Livzon | LZM-009 | PD1 |
| Jounce | pimivalimab | PD1 |
| Shangdong | rulonilimab | PD1 |
| Novartis | spartalizumab | PD1 |
| Tayu | TY-101 | PD1 |
| 3SBio | 609A | PD1 |
| Bio-Thera | BAT-1308 | PD1 |
| Generium | GNR-051 | PD1 |
| Suzhou | hAB21 | PD1 |
| Lyvgen | LVGN-3616 | PD1 |
| Mabwell | MW-11 | PD1 |
| Servier/Symphogen | Sym-021 | PD1 |
| Synamore | SYN-125 | PD1 |

TABLE 3

| Company | Generic Drug Name | Target |
|---|---|---|
| Roche | atezolizumab | PDL1 |
| Merck KGaA | avelumab | PDL1 |
| AZ | durvalumab | PDL1 |
| Alphamab | envafolimab | PDL1 |
| Cstone | sugemalimab | PDL1 |
| Atridia | adebrelimab | PDL1 |
| Klus | KL-A167 | PDL1 |
| Lee's | socazolimab | PDL1 |
| CBT | APL-502 | PDL1 |
| TG | cosibelimab | PDL1 |
| ImmuneOncia | IMC-001 | PDL1 |
| Adagene | ADG-104 | PDL1 |
| Genrix | GR-1405 | PDL1 |
| Zhejiang Hisun | HS-636 | PDL1 |
| Laekna | LAE-005 | PDL1 |
| Lepu | LP-002 | PDL1 |
| Cytomx | pacmilimab | PDL1 |
| Abbisko | ABSK-043 | PDL1 |
| Jiangsu Hyamab | | PDL1 |
| Biocad | BCD-135 | PDL1 |

Several of these anti-PD1 antibodies are described the U.S. patent documents. For example: U.S. Pat. Nos. 8,008,449, 8,168,757, 9,205,148, 9,987,500, 9,815,897, 11,414,487, 10,294,299, 11,034,765, 10,239,942, 10,323,091, US20190225689, US20190270817, U.S. Pat. No. 9,914,783, CN106432494, U.S. Pat. No. 11,028,169.

Several of these anti-PDL1 antibodies are described the U.S. patent documents. For example: U.S. Pat. Nos. 9,920,123, 9,938,345, 7,943,743, 8,217,149, 9,624,298, 10,336,824, 11,274,153, 10/336,823.

### Nucleic Acids

Embodiments of the disclosure include a polynucleotide encoding the single immunoglobulin variable domain or the heterodimeric molecule as described herein.

### EXAMPLES

The Examples that follow are illustrative of specific embodiments of the disclosure, and various uses thereof. They are set forth for explanatory purposes only, and should not be construed as limiting the scope of the invention in any way.

### Example 1: Discovery of Anti-IL2Rgamma, Anti-IL2Rbeta and Anti-CD8 VH Domains

Discovery of IL2Rgamma, IL2Rbeta and CD8 binders is performed using the HuTARG™ mammalian display system (Kielczewska, A. et al. Development of a potent high-affinity human therapeutic antibody via novel application of recombination signal sequence-based affinity maturation. J Biol Chem 298, 101533, doi:10.1016/j.jbc.2021.101533 (2022). Briefly, the most predominate 47 functional human germline IGHV segments are included within the HuTARG library. VDJ recombination is used to generate a large repertoire of novel VH domains (no light chain) that get displayed as membrane tethered proteins. IL2Rgamma (CD132) and IL2Rbeta (CD122) extracellular domains were produced in house as rabbit or mouse Fc fusion proteins. A CD8a/b soluble heterodimeric extracellular domain protein was obtained from R&D Systems (catalog number 9358-CD-050) and biotinylated using standard kits and lysine chemistry. The mammalian display library was expanded and enriched for binding to these antigens by flow cytometry using alternating rounds of anti-mouse IgG PE or anti-rabbit IgG Alexa Fluor 647 (for IL2Rgamma or IL2Rbeta) or alternating rounds of streptavidin Brilliant Violet 421, streptavidin Alexa Fluor 647 or NeutrAvidin DyLight 650 (for biotinylated CD8a/b) as detection reagents. Three to four rounds of FACS enrichment followed by expansion were needed to obtain a solid pool of VH binders whose sequences could be identified using next generation sequencing (NGS) on a MiSeq device (Illumina).

### Example 2: Identification of Functional IL2Rgamma/IL2Rbeta Agonists with Conditional Activity Reliant on the Binding of a CD8

Twenty-five anti-IL2Rgamma, two anti-IL2Rbeta, and eighteen anti-CD8 VH domains are combined into multiple homodimeric Fc constructs (multi-specific molecules) with adjacent anti-IL2Rgamma/IL2Rbeta VH domains linked using a glycine/serine linker either N-terminally or C-terminally to anti-CD8 VH domains N-terminal to the IgG1 Fc hinge. In a separate design, the CD8 VH domains are fused recombinantly to the C-terminus of the IgG1 Fc using a glycine/serine linker. In another design, the CD8 VH domain is located between the anti-IL2Rgamma VH domain and the anti-IL2Rbeta VH domain. The VH domain subunits are cloned to evaluate the impact of multiple glycine/serine linker lengths ranging from 5 to 25 amino acids. Both orientations (IL2Rgamma VH-linker-IL2Rbeta VH and IL2Rbeta VH-linker-IL2Rgamma VH) are included in the library. Greater than 300,000 individual multi-specific expression constructs are stably integrated into the HuTARG™ cell line (1 molecule construct per cell) harboring human IL2Rgamma, human IL2Rbeta, human CD8a controlled by an inducible promotor and a reporter gene (green fluorescence protein—GFP) under pSTAT5 promotor control. HuTARG cells are sorted for GFP by flow cytometry under conditions where CD8 expression is upregulated by an inducer. This pool of GFP+ cells is expanded, sorted again both under conditions of CD8a induction or not, lysed and is subjected to NGS using a MiSeq sequencer. Multi-specific molecule sequences from cells that are GFP+ only under conditions of CD8-induction are subject to additional characterization.

### Example 3: Construction of a LAG3 Inhibitor with Highly Conditional LAG3-Dependent IL2Rgamma/Beta Activity

The CD8-conditional IL2Rgamma/beta agonist subunits were fused to one arm of a LAG3 inhibitor antibody via standard recombinase-based subcloning. The first chain in this anti-LAG3 heavy chain comprised a single anti-IL2Rgamma VH (SEQ ID NO: 1)/linker (PPGG SEQ ID NO: 52)/anti-IL2Rbeta VH (SEQ ID NO: 2) agonistic subunit recombinantly fused to the N-terminus of the anti-LAG3 inhibitor antibody heavy chain (SEQ ID NO: 3) through a linker (PPGGQGQGGQGGGQGGG SEQ ID NO: 68), which anti-LAG3 inhibitor antibody heavy chain is fused to a human IgG1 constant region; heavy chain combined as SEQ ID NO: 48). The human IgG1 constant region comprised 3 modifications to eliminate FcGammaR-binding and C1q(complement)-binding (L234A, L235A, K322A—EU numbering) and 3 modifications in the CH3 domain to induce heavy chain heterodimerization (K370E, K409D, K439E). The second heavy chain comprises only the LAG3 inhibitor heavy chain (SEQ ID NO: 3) on an IgG1 backbone containing the same modifications to eliminate FcGammaR- and C1q binding, but with complementary modifications (E356K, E357K, D399K) in the CH3 domain to enable proper heterodimerization with the first antibody heavy chain (molecule combined as SEQ ID NO: 49). The third antibody chain is the LAG3 inhibitor kappa light chain (SEQ ID NO: 9).

TNRX-257 protein was generated by co-expressing SEQ ID NO: 48, SEQ ID NO: 49, and SEQ ID NO: 9 together in mammalian cells as described below. A schematic diagram of the assembled protein is shown in FIG. 1.

Two additional molecules contain the anti-IL2Rgamma VH-linker-anti-IL2Rbeta subunit linked directly to the antibody Fc with a heterodimer motif. These chains were paired with the anti-LAG3 heterodimer chain (SEQ ID NO: 49) and anti-LAG3 light chain (SEQ ID NO: 9) to generate multi-specific LAG3 inhibitor molecules with LAG3-conditional IL2Rgamma/beta activation (FIG. 2). The first chain used in a heterodimer combined with SEQ ID NO: 49 comprised a single anti-TL2Rgamma VH (SEQ ID NO: 1)/linker (GGGSGS SEQ ID NO: 56)/anti-IL2Rbeta VH (SEQ ID NO: 2) agonistic subunit recombinantly fused to the N-terminus of the antibody Fc (SEQ ID NO: 44) through a linker (PP); the combined molecule is shown as SEQ ID NO: 50). The second chain used in a heterodimer combined with SEQ ID NO: 49 comprised a single anti-TL2Rgamma VH (SEQ ID NO: 1)/linker (PPGG SEQ ID NO: 52)/anti-IL2Rbeta VH (SEQ ID NO: 2) agonistic subunit recombinantly fused to the N-terminus of the antibody Fc (SEQ ID NO: 44) through a linker (PP); the combined molecule is combined shown as SEQ ID NO: 51). The two versions of this molecule include variation in the linker between the anti-IL2Rgamma VH and anti-IL2Rbeta VH.

The complete amino acid sequences each of SEQ ID NOS: 48, 49, 50, and 51 appear at the end of the specification.

### Example 4: Developability and Affinity Optimization of the Anti-IL2Rbeta VH Domain

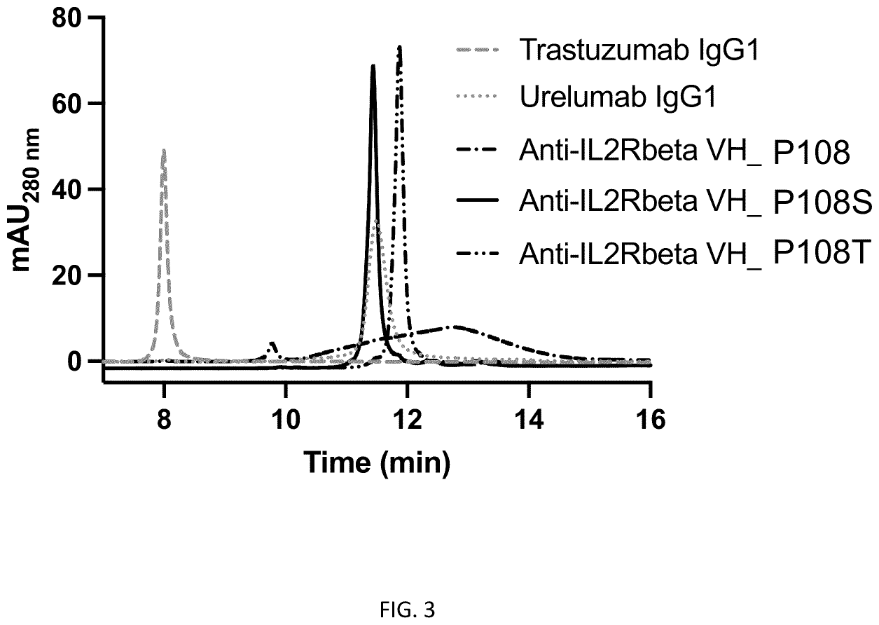

The initially discovered anti-IL2Rbeta VH domain, when expressed as an N-terminal VH domain fusion to IgG1-Fc, contained a proline at position 100c in HCDR3 that led to strong hydrophobic character based on a strong and broad binding profile on a HIC butyl analytical HPLC column (Thermo Fisher) using a Vanquish Flex HPLC (Thermo Fisher). Experimental conditions were provided by the column manufacturer (Thermo Fisher). Two modifications, P100cS and P100cT (Kabat numbering), were found by screening multiple variants within the anti-IL2Rbeta VH domain that significantly reduced hydrophobicity (FIG. 3). The P100cS modification was within, e.g., SEQ ID NO: 2, SEQ ID NO: 48, SEQ ID NO: 50 and SEQ ID NO: 51.

Modifications to increase the potency of the anti-IL2Rbeta VH towards IL2Rbeta were made in the anti-IL2Rbeta VH HCDR1 and HCDR2 using the multi-specific format including anti-LAG3 and a single anti-IL2Rgamma/anti-IL2Rbeta unit. The anti-IL2Rbeta VH domain modifications were generated as eBlocks (Integrated DNA Technologies) and cloned using the Infusion recombinase protocol (Takara Bio) into the multi-specific format. The variants were expressed in the Expi293 system (Thermo Fisher) in 24-well microwell format. Their binding potency was assessed using an enzyme-linked immunosorbent assay (ELISA). Briefly, 96 well ELISA plates (high protein binding Greiner Microlon) were coated overnight at 4° C. with a polyclonal anti-human IgG-Fc antibody diluted 1:1000 in PBS buffer (Jackson Immunoresearch). The plate was then washed with PBST and blocked using 5% BSA in PBS for 1 hour at room temperature (RT). The BSA was washed off and the anti-IL2R VH-Fc variants were added at concentrations ranging from 5 to 20 mg/L in a solution containing PBS/0.1% Tween 20/1% BSA. After a 1 hour incubation, the multi-specific variants were washed off, followed by a 1 hour incubation of human IL2Rbeta-Avi protein (R&D Systems). The IL2Rbeta protein was then washed off, followed by incubation with Streptavidin HRP (Jackson Immunoresearch), another wash, and then detection using 3,3′,5,5′-Tetramethylbenzidine (TMB) substrate. After roughly 10-20 minutes, the 96 well plates were neutralized with 0.2 M H₃PO₄ and absorbance was read at 425 nm using a iD5 plate reader (Molecular Devices). Multiple modifications from the germline HCDRs induced improved binding affinity based on ELISA measurements including D31R, D31K, D31S, D31Y, Y32F, Y33F, E531, E53S, and E53Y (Table 3). The D31R modification was chosen as part of, e.g., SEQ ID NO: 2, SEQ ID NO: 48, SEQ ID NO: 50 and SEQ ID NO: 51.

TABLE 3
ELISA EC50 values for anti-IL2Rbeta
VH HCDR1 and HCDR2 variants

| Anti-IL2Rbeta VH | ELISA EC₅₀ |
|---|---|
| Modification | (ug/mL) |
| Wild-type | 0.65 |
| D31K | 0.40 |
| D31R | 0.13 |
| D31S | 0.21 |
| D31Y | 0.24 |
| Y32F | 0.24 |
| Y33F | 0.24 |
| E53I | 0.30 |
| E53S | 0.30 |
| E53Y | 0.40 |

### Example 5: Affinity Optimization of the Anti-LAG3 Inhibitor Antibody Light Chain

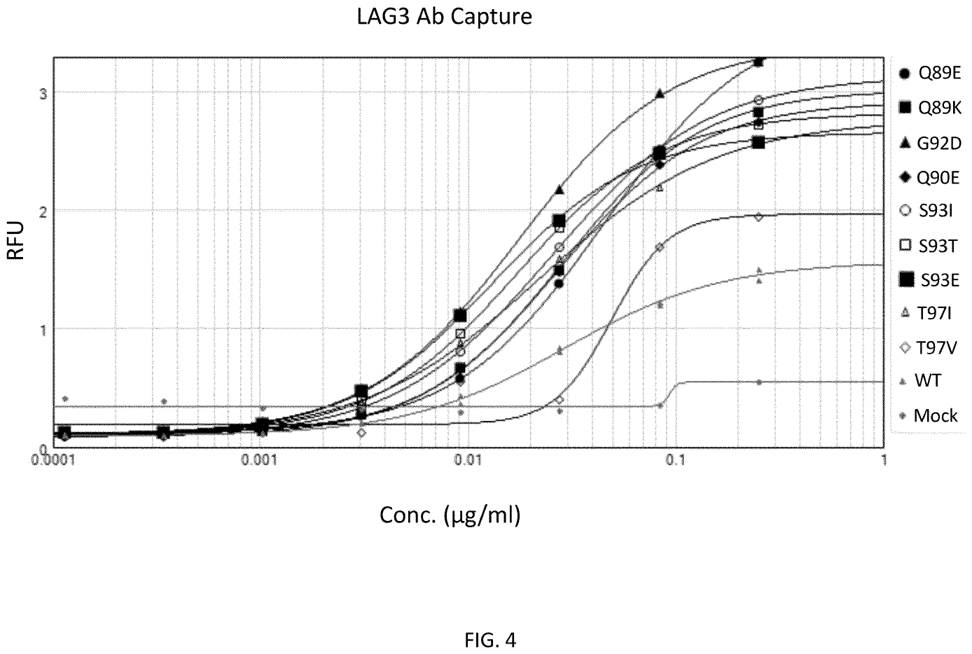

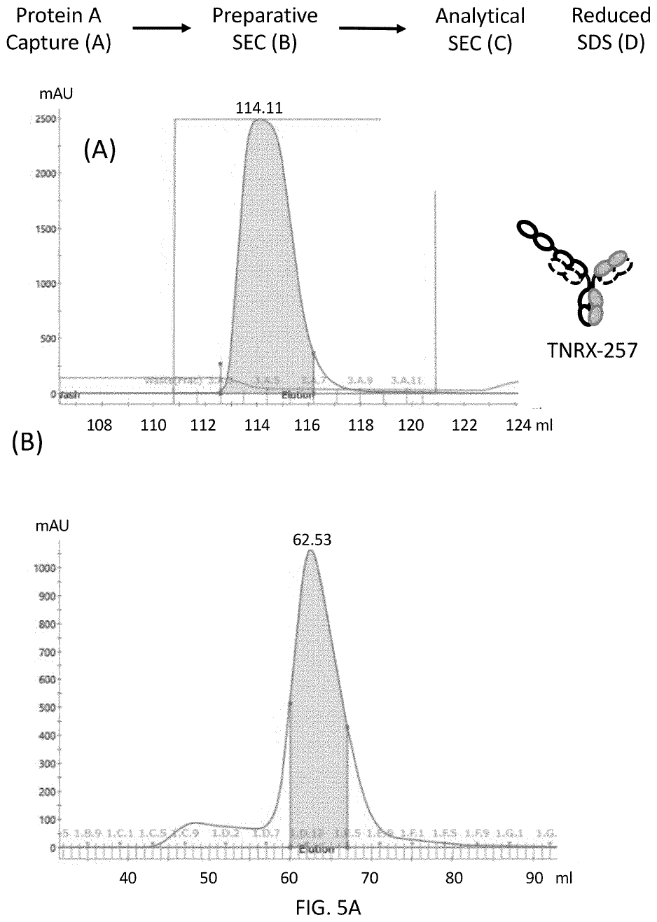

A library of single mutant variants within the anti-LAG3 VL complementarity determining region 3 (CDR3) was cloned and screened for improved binding to soluble LAG3 antigen using an ELISA method. Briefly, the variable light chain domain from the antibody fragments were synthesized by IDT as eblocks with unique single point mutations in CDR L1 and CDR L3. Eblocks are subcloned into a mammalian expression vector for full-length human IgG expression as heavy chain+light chain multi-specifics including the anti-IL2Rgamma/anti-IL2Rbeta VH subunit and expressed using the Expi293 system (Thermo Fisher) in 24-well microwell format. Supernatants (2 mL) containing the variants were harvested after 5 days culture using standard protocols from Thermo Fisher. The variant supernatants were assessed for their binding using an ELISA. The variants were captured using an anti-human Kappa antibody (Southern Biotech) adsorbed on high protein binding polystyrene 96 well plates, washed, blocked with BSA in PBS, and incubated with LAG3 antigen. After an additional wash step and incubation with anti-antigen HRP secondary, binding levels were detected with TMB (OD425 nm measured after addition of stop solution). Multiple modifications were found that increase binding of the LAG3 antibody to soluble LAG3 including Q89E, Q89K, G92D, Q90E, S93I, S93T, S93E, T97V and T97I (FIG. 4). The S93E modification was chosen within the final anti-LAG3 light chain (SEQ ID NO: 8 and 9).

### Example 6: Expression and Purification of LAG3i-IL2R Multi-Specific Molecules

For the experiments described below, TNRX-257, TNRX-190, control proteins, and libraries used to improve various lead protein properties are all produced using the Expi293™ transient expression system using protocols developed by the manufacturer (Thermo Fisher).

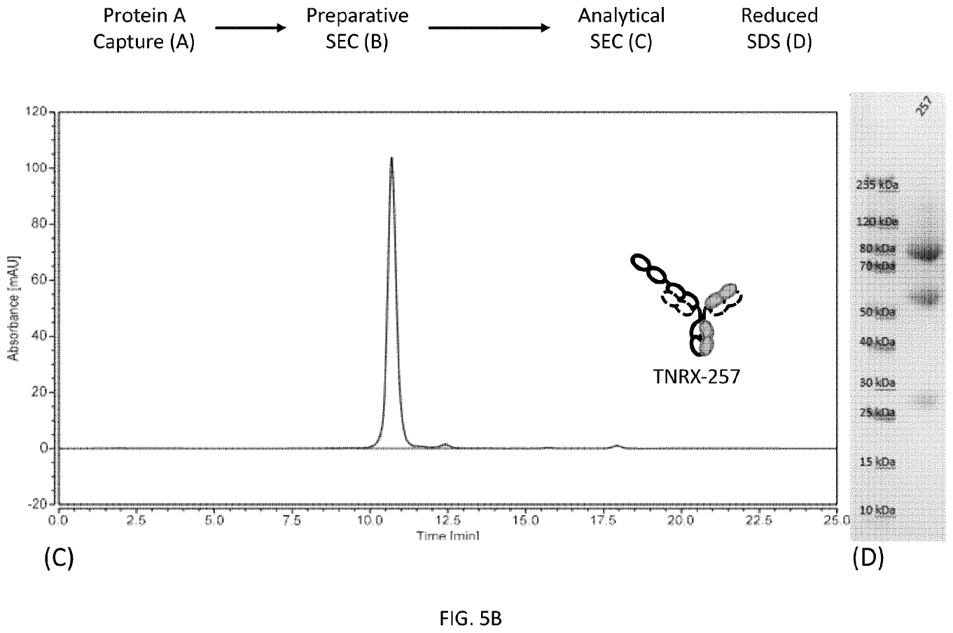

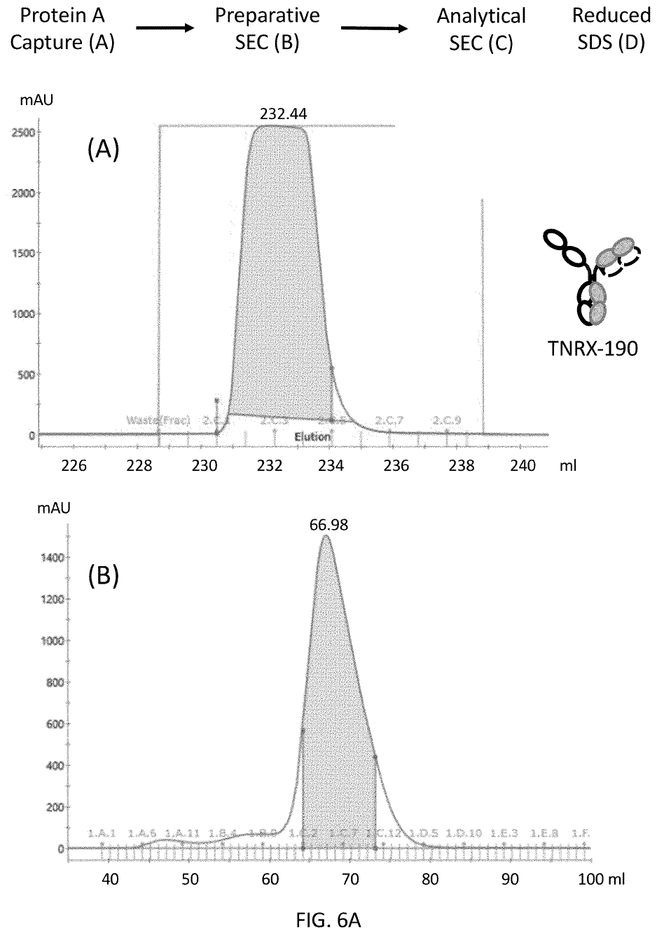

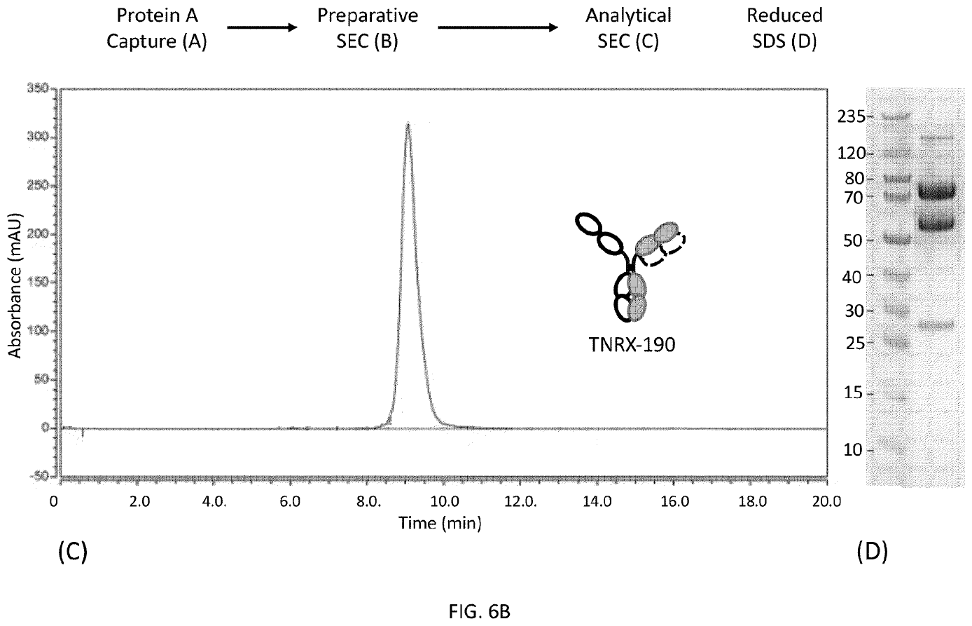

For TNRX-257 and TNRX-190 production, three DNA expression plasmids encoding the TNRX-257 multi-specific molecule or the TNRX-190 multi-specific molecule were transfected into Expi293 HEK cells in Expi293 Media. Cells were grown at 37° C. with 8% CO₂ with shaking for 5 days. After 5 days, cells were pelleted via centrifugation, and the supernatant was harvested and clarified using sterile PES membranes. Clarified supernatant was loaded onto a HiTrap MabSelect PrismA protein A column. The column was washed with buffer, and TNRX-257 or TNRX-190 was eluted in a single-step at low pH and immediately neutralized. Fractions from the primary peak of the chromatogram were pooled, filtered via a cellulose acetate membrane, and quantified through absorbance at 280 nm. Isolated TNRX-257 and TNRX-190 multi-specific molecules were then loaded onto a preparative size exclusion column composed of Superdex 200 pg resin equilibrated in PBS and run in an isocratic protocol. Fractions from the primary peak of the chromatogram were pooled, filtered via cellulose acetate membrane, and quantified through absorbance at 280 nm. The purity of LAG3i-IL2Rγβ proteins was confirmed though use of an HPLC equipped with a TOSOH TSKgel G3000SWxl size exclusion column and determining the area under the curve monitored at 280 nm. SDS-PAGE was then performed under denaturing as well as reducing and non-reducing conditions to assess TNRX-257 and TNRX-190 composition (FIGS. 5A, 5B, 6A, and 6B).

### Example 7: TNRX-257 Blocks the Interaction of LAG3 with MHC Class II

Two assays were developed to characterize TNRX-257's ability to block LAG3's interaction with MHC Class II and the suppressive activity of LAG3.

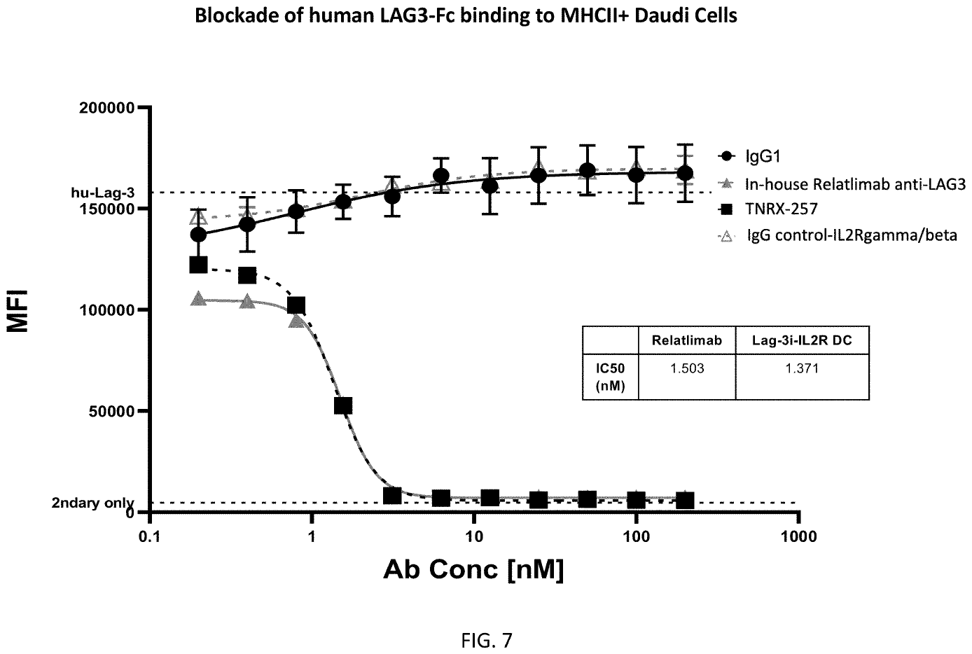

First, an assay was developed to assess the ability of the LAG3i-IL2Rgamma/beta agonist to block binding of soluble LAG3-mouse IgG-Fc fusion protein to MHC class II expressed on Daudi cells. Binding of hLAG3-mouse IgG-Fc fusion protein to the Daudi cells was detected using a PE Goat anti-mouse IgG antibody. Data were analyzed with the Agilent NovoCyte Advanteon flow cytometer and shown as mean fluorescent intensity, percentage, and IC₅₀. Each sample was assayed in duplicate. The assay demonstrated that TNRX-257 inhibited binding of soluble LAG3-Fc to MHC class II on Daudi cells with a similar IC₅₀ as in-house derived relatlimab (25F7, WO2010019570) that was used as positive control (FIG. 7).

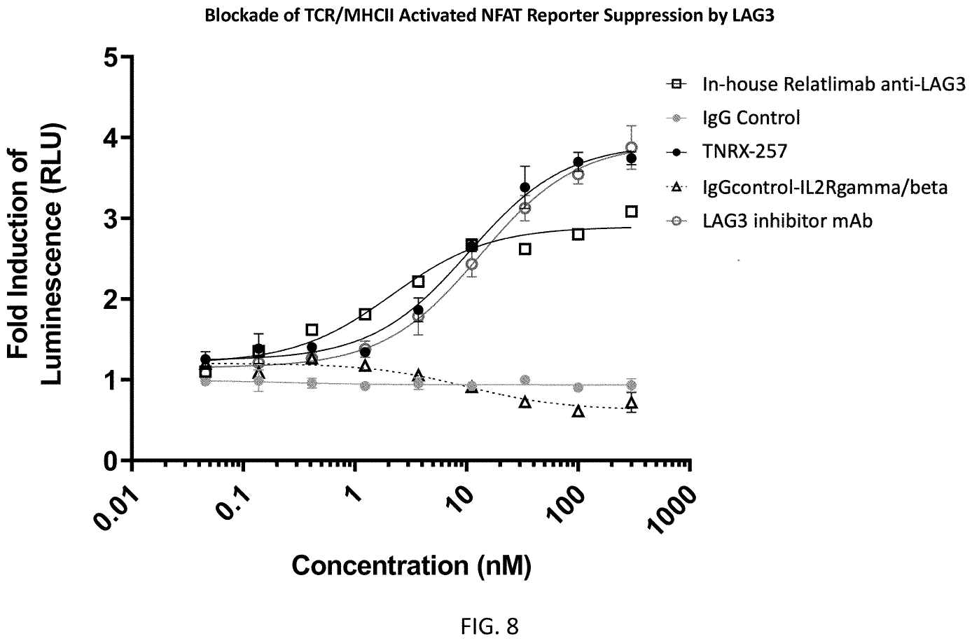

Next, a LAG3-specific NFAT reporter assay was used to evaluate the ability of the TNRX-257 to block the ability of LAG3 to suppress T cell receptor signaling. The data show that TNRX-257, the LAG3i mAb control, and the in-house relatlimab IgG control target and block the LAG3/MHCII interaction resulting in increased luciferase reporter activity driven by T-cell activation pathway-dependent response elements genetically engineering into the Jurkat effector cells (FIG. 8). The Isotype control and an untargeted IgG-IL2Rgamma/beta control do not block the LAG3/MHCII interaction (FIG. 8). The LAG3/MHCII Blockade Bioassay purchased from Promega (#JA1115).

### Example 8: Highly Conditional STAT5 Activation of LAG3+ Cells by TNRX-257

IL2-mediated IL2Rgamma/beta signaling leads JAK1/JAK3 recruitment and phosphorylation of the STAT5 protein that facilitates many of the downstream biological activities of IL2. The LAG3i-IL2R multi-specific's ability to produce pSTAT5 is measured on primary CD8+ T cells. Briefly, PBMCs were isolated from whole blood collected at the San Diego blood bank and frozen. After thawing, the PBMCs were stimulated for 48 hours in a flask coated with anti-CD3 (OKT3) and with soluble anti-CD28 to induce LAG3 expression, then rested overnight. Activation of the PBMCs by the LAG3i-IL2R multi-specific, human IL2, or an IgG control were performed for 15 minutes, then the cells were fixed with BD CytoFix and stained for pSTAT5 using pY694 (BD). Discrimination of CD8+ T cells was performed using anti-CD3 (SK7-PE), anti-CD8 (SK1-BV650) both from Biolegend. Discrimination of LAG3-expressing cells was performed by staining for LAG3 using an antibody (3DS223H-AF647, Invitrogen/eBiosciences) that does not interfere with the LAG3i-IL2R multi-specific. Flow cytometric analyses was performed on a Novocyte3000.

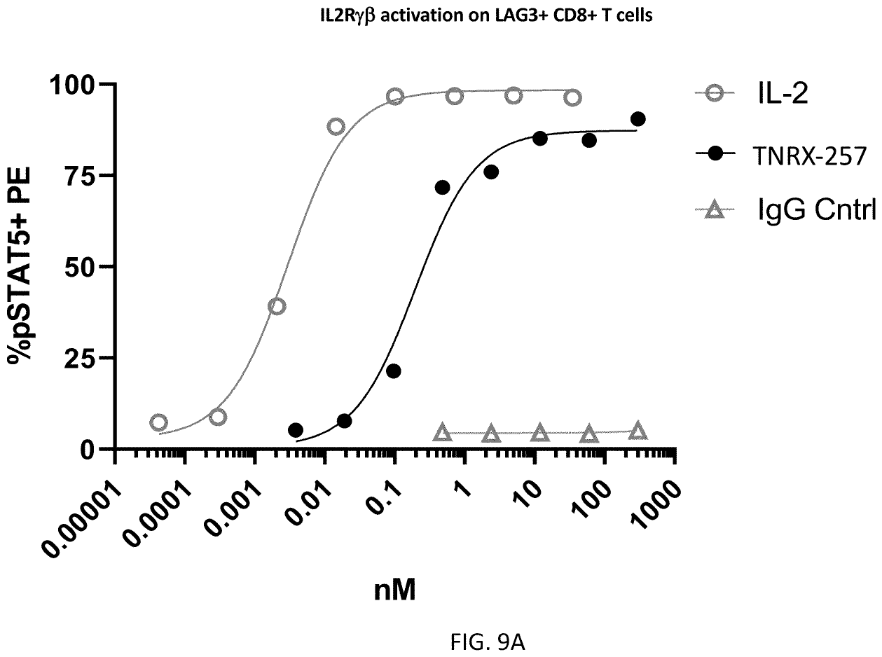

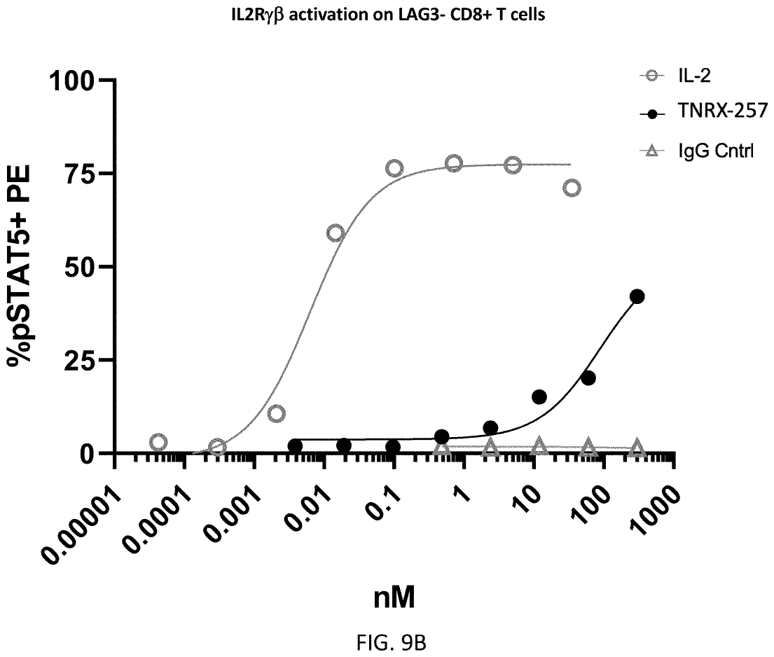

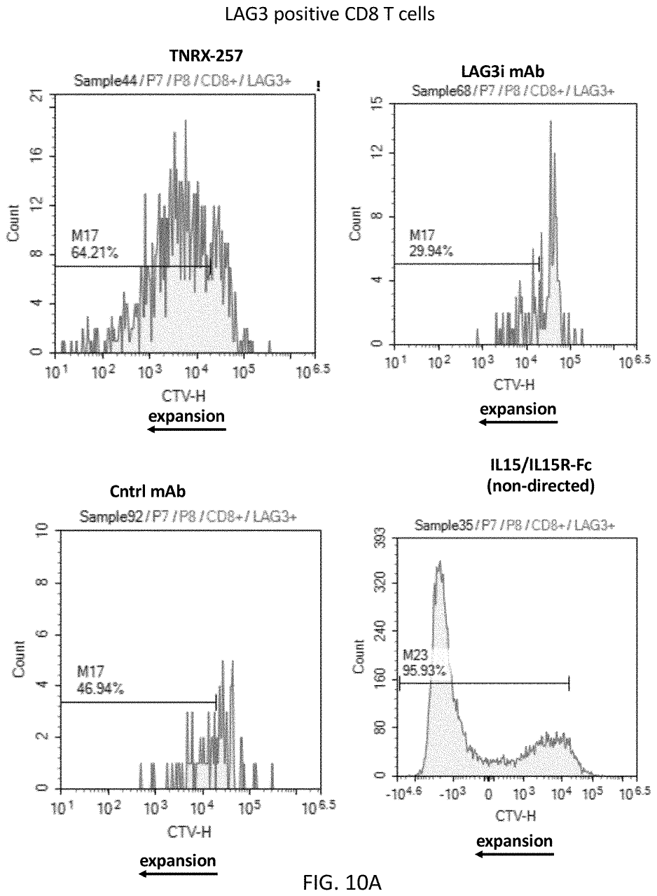

TNRX-257 led to highly conditional activation of LAG3+ CD8+ T cells (FIG. 9A) compared to LAG3−CD8+ T cells (FIG. 9B) with over 1000-fold specificity for cells expressing LAG3. Alternately, human IL2 indiscriminately activated CD8+ T cells regardless of LAG3's expression.

### Example 9: TNRX-257 Induces LAG3-Specific Expansion of Metastatic Melanoma TILs

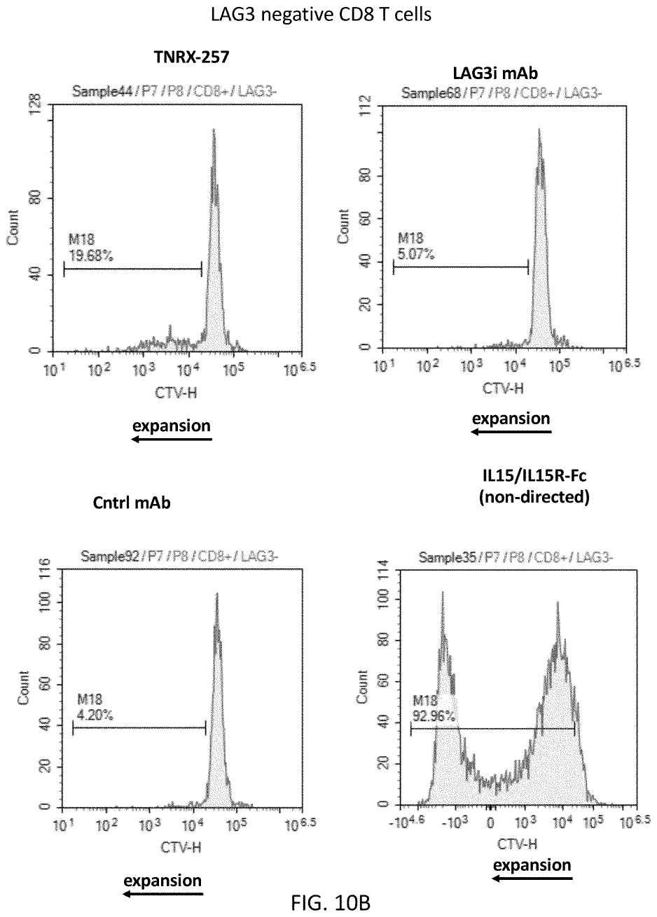

Tissue samples were provided by the Cooperative Human Tissue Network (CHTN), which is funded by the National Cancer Institute. Specifically, human metastatic melanoma biopsies obtained from CHTN were processed into single cell suspension and cryopreserved. These cells were revived, labelled with Cell Trace Violet (CTV) and cultured with TNRX-257, LAG3i mAb, control mAb and an IL15/IL15R non-directed agonist controls for 5 days. The cells were stained for immune cell surface and activation markers. Cell proliferation was analyzed based on dilution of CTV signal over time. TNRX-257 selectively induced proliferation of LAG3+ CD8 T cells (FIG. 10A) but not proliferation of LAG3− CD8 T cells (FIG. 10B). In contrast, IL15/IL15R non-directed agonist induced proliferation of both LAG3+(FIG. 10A) as well as LAG3− CD8 T cells (FIG. 10B). LAG3i mAb, control mAb did not induce marked proliferation of CD8 T cells regardless of LAG3 expression. These results show remarkable selectivity of TNRX-257 in inducing CD8 T cell proliferative responses in human TILs of metastatic melanoma.

### Example 10: TNRX-257 Synergizes with PD1 Inhibitor to Induce IFNgamma Release from TILs

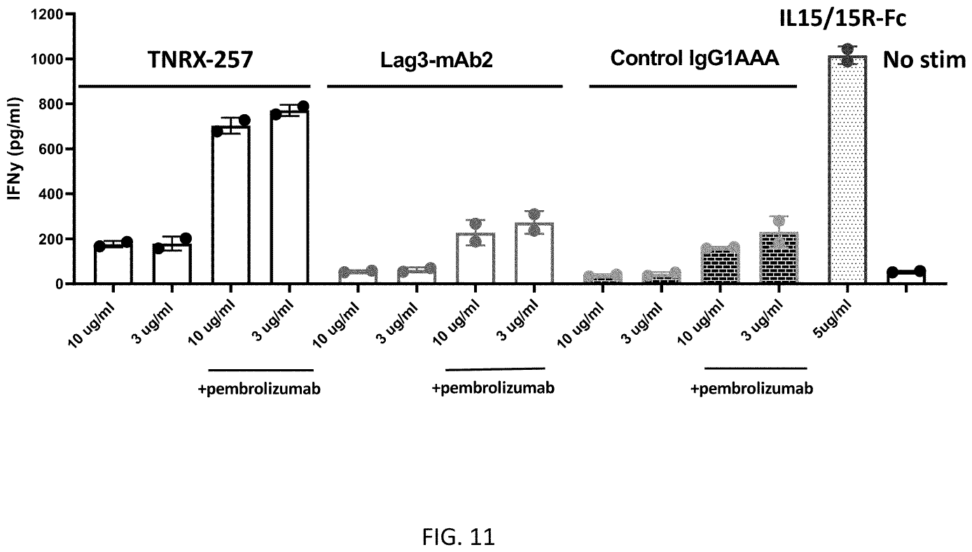

The ability of TNRX-257 to activate tumor infiltrating lymphocytes (TILs) from metastatic melanoma biopsies was assessed by its ability to induce the secretion of the inflammatory cytokine, IFNgamma. Human metastatic melanoma single cells were cultured with TNRX-257, LAG3i mAb, control mAb in presence or absence of pembrolizumab (anti-PD1) and an IL15/IL15R-Fc non-directed agonist control (as described in previous example) for 5 days. Culture supernatants were collected on day 3, and IFN-gamma was analyzed by MSD. TNRX-257 by itself caused release of IFNgamma that was synergistically enhanced when combined with pembrolizumab, whereas, the LAG3i mAb by itself did not induce IFNgamma and did not synergize with pembrolizumab as shown by combination group of control mAb+pembrolizumab (FIG. 11). Thus, it was demonstrated that LAG3i/IL2R DC promoted IFNgamma and synergized with pembrolizumab to further increase IFNgamma production by human metastatic melanoma TILs.

### Example 11: TNRX-257 Induces Strong Anti-Tumor Immunity In Vivo

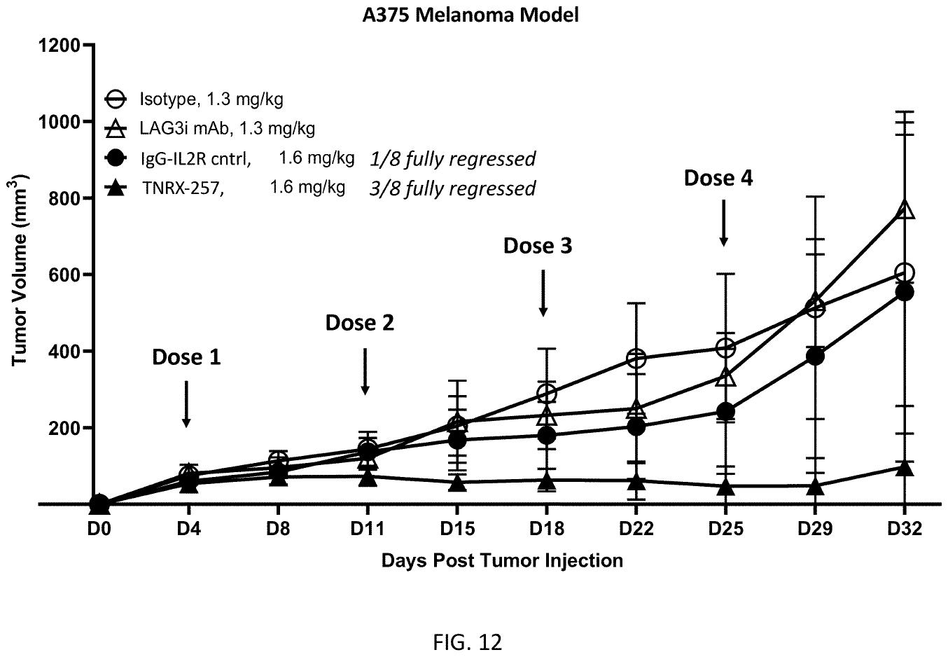

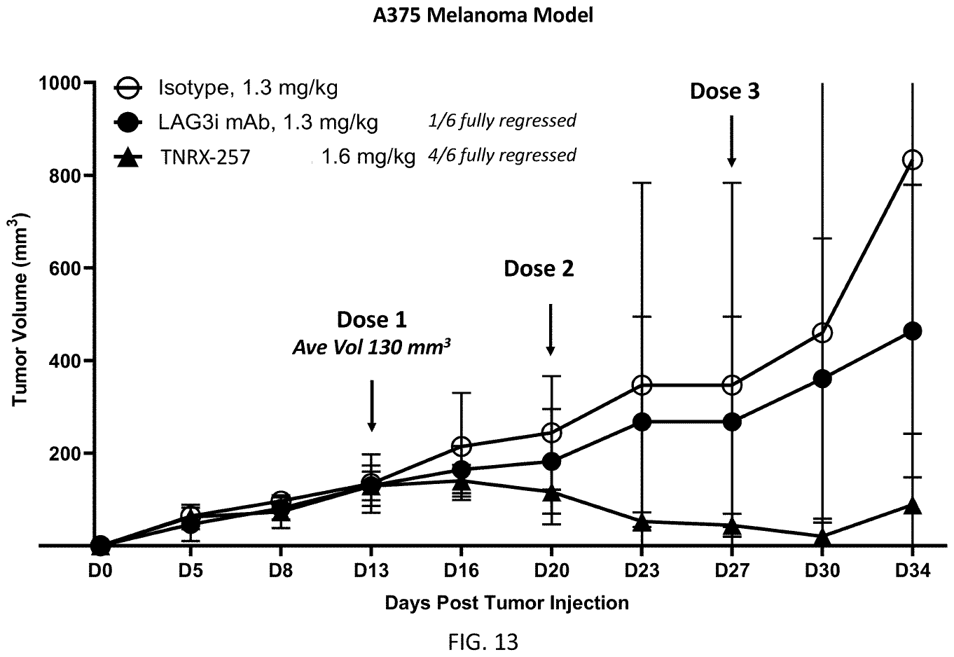

NSG-MHC I/II DKO mice (Jackson Laboratories, Strain 025216) were engrafted with 5 million CMVpp65 expressing A375 cells (ATCC, CRL-1619) in their right flank (subcutaneous) and 10 million CMV reactive human PBMCs (retro-orbital). Dosing with TNRX-257 or control molecules was administered (intraperitoneal) every 7 days beginning on day 4 (FIG. 12) or 13 (FIG. 13) post tumor and PBMC engraftment. Weight and tumor volume were monitored throughout the study period. In the early dosing A375 melanoma model (FIG. 12, n=8 per group), mice treated with TNRX-257 showed robust tumor growth inhibition (significant by day 18, p=0.013 Isotype vs TNRX-257, 2-way ANOVA with Tukey multiple comparison test). Three of the eight mice exhibited complete tumor regression. Treatment with either (1) the IgG-IL2R comprising the anti-IL2Rgamma/IL2Rbeta subunit in a similar orientation as TNRX-257 fused to a control non-binding antibody, or (2) the LAG3i mAb of TNRX-257 (no anti-IL2R VHs) did not have a significant impact on tumor growth as compared to the IgG control (p=ns, 2-way ANOVA with Tukey multiple comparison test). This indicates that combination of the LAG3 targeting with IL2Rγ/β agonism was necessary for the potent therapeutic efficacy that the TNRX-257 exhibited in this model (p<0.0001 compared to all controls by day 32, 2-way ANOVA with Tukey multiple comparison test). For the established tumor model (FIG. 13), A375 melanoma tumors grew in vivo to an average volume of 130 mm³ prior to dose initiation. Mice were then randomized into treatment groups (n=7 per group). Tumor growth inhibition was seen in the TNRX-257 cohort by seven days post dose one and became significantly different from Isotype by day 30 (p=0.01, p<0.0001 day 34, 2-way ANOVA with Tukey multiple comparison test). Four of the seven mice treated with TNRX-257 had complete tumor regression. This is compared to only one tumor regression event in the LAG3i mAb cohort (p=0.04 between LAG3i mAb and both Isotype and LAG3i/IL2R DC at day 34, 2-way ANOVA with Tukey multiple comparison test) and none in the IgG isotype group. This demonstrated that TNRX-257 has strong induction of tumor regression when challenged with an established tumor.

### Example 12: TNRX-257 Fc Effector, Fc Heterodimerization, and Linker Permutations

To explore various permutations of TNRX-257 using different Fe effector mutations to reduce FcγR/C1q, different Fe heterodimerization designs to induce correct chain pairing, and a different 17 amino acid linker between the anti-IL2Rbeta VH binding domain and anti-LAG3 Fab domain within the agonist arm of the Tentacle, multiple variants of TNRX-257 were made and tested to determine the impact on expression, purity, and activity. The variants include the following:
(Variant 1) TNRX-257_AA with K322;
(Variant 2) TNRX-257_GS_AA_KH using Knobs-in-Holes [T366W (knob) with Y349C and T366S/L368A/Y407V (hole) with S354C where the two engineered cysteines form a stabilizing intermolecular disulfide bond] mutations to drive heterodimerization, a 17 amino acid Gly/Ser linker between the anti-TL2Rbeta VH and the LAG3 mAb2 VH, and K322;
(Variant 3) TNRX-257_GS_KH using Knobs-in-Holes mutations to drive heterodimerization, and a 17 amino acid Gly/Ser linker between the anti-IL2Rbeta VH and the LAG3 mAb2 VH;
(Variant 4) TNRX-257_KH using Knobs-in-Holes mutations to drive heterodimerization;
(Variant 5) TNRX-257_DS_N297G with N297G to eliminate glycosylation, and a stabilizing disulfide bond within the CH2 domain.

Sequences of the permutants follow:

(Variant 1) TNRX-257_AA (HC Het1)
(SEQ ID NO: 86)
EVQLVESGGGVVRPGGSLRLSCCASGFTEDDYGMSWVRRAPGKGLEWVAGINWNGGSTGYADSVKGRF
TISRDNEKNCLYLQMNSLRAEDTALYHCASATSGTAFDIWGQGTMVTVSSPPGGEVQLVQSGAEVKKP
GACVKISCKVSGYTFTRYYMHWVQRAPGKGLEWIGLVDPEDGETIYAEKFQGRVTITADTSTDTAYME
LCSLRSEDTAVYYCATDFVKLDESLSFQHWGQGTLVTVSSPPGGQGQGGQGGGQGGGQVQLVESGGGV
VQPGRSLRLSCAASGFTFSSYGMHWVRQAPGKGLEWVAVIWYDGSNKYYADSVKGRFTISRDNSKNTL
YLQMNSLRAEDTAVYYCAREWAVASWDYGMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVELFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREP
QVYTLPPSREEMTKNQVSLTCLVEGFYPSDIAVEWESNGQPENNYKTTPPVLDSDGSFFLYSDLTVDK
SRWQQGNVFSCSVMHEALHNHYTQESLSLSPGK
(Variant 1) TNRX-257_AA (HC Het2)
(SEQ ID NO: 87)
QVQLVESGGGVVQPGRSLRLSCAASGFTFSSYGMHWVRQAPGKGLEWVAVIWYDGSNKYYADSVKGRF
TISRDNSKNTLYLQMNSLRAEDTAVYYCAREWAVASWDYGMDVWGQGTTVTVSSASTKGPSVFPLAPS
SKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPEAAGGPSVFLFPPKPKDTLMISRTPEVTCVVVDVS
HEDPEVKENWYVDGVEVHNAKTKPREEQYNSTYRVVSVLTVLHQDWLNGKEYKCKVSNKALPAPIEKT
ISKAKGQPREPQVYTLPPSRKKMTKNQVSLTCLVKGFYPSDIAVEWESNGQPENNYKTTPPVLKSDGS
FFLYSKLTVDKSRWQQGNVFSCSVMHEALHNHYTQKSLSLSPGK
TNRX-257_AA (LC)
(SEQ ID NO: 9)
EIVLTQSPGTLSLSPGERATLSCRASQSVSSSYLAWYQQKPGQAPRLLIYGASSRATGIPDRESGSGS
GTDFTLTISRLEPEDFAVYYCQQYGESPFTFGPGTKVDIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSS
PVTKSENRGEC
(Variant 2) TNRX-257_GS_AA_KH (HC Het1)
(SEQ ID NO: 89)
EVQLVESGGGVVRPGGSLRLSCCASGFTEDDYGMSWVRRAPGKGLEWVAGINWNGGSTGYADSVKGRF
TISRDNEKNCLYLQMNSLRAEDTALYHCASATSGTAFDIWGQGTMVTVSSPPGGEVQLVQSGAEVKKP
GACVKISCKVSGYTFTRYYMHWVQRAPGKGLEWIGLVDPEDGETIYAEKFQGRVTITADTSTDTAYME
LCSLRSEDTAVYYCATDFVKLDESLSFQHWGQGTLVTVSSPPGGSGGGGSGGGGSGGQVQLVESGGGV
VQPGRSLRLSCAASGFTFSSYGMHWVRQAPGKGLEWVAVIWYDGSNKYYADSVKGRFTISRDNSKNTL
YLQMNSLRAEDTAVYYCAREWAVASWDYGMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREP
QVCTLPPSREEMTKNQVSLWCLVKGFYPSDIAVEWESNGQPENNYKTTPPVLDSDGSFFLYSKLTVDK
SRWQQGNVFSCSVMHEALHNHYTQKSLSLSPGK
(Variant 2) TNRX-257_GS_AA_KH (HC Het2)
(SEQ ID NO: 90)
QVQLVESGGGVVQPGRSLRLSCAASGFTESSYGMHWVRQAPGKGLEWVAVIWYDGSNKYYADSVKGRF
TISRDNSKNTLYLQMNSLRAEDTAVYYCAREWAVASWDYGMDVWGQGTTVTVSSASTKGPSVFPLAPS
SKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPEAAGGPSVELFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKT
ISKAKGQPREPQVYTLPPCREEMTKNQVSLSCAVKGFYPSDIAVEWESNGQPENNYKTTPPVLDSDGS
FFLVSKLTVDKSRWQQGNVFSCSVMHEALHNHYTQKSLSLSPGK
TNRX-257_GS_AA_KH (LC)
(SEQ ID NO: 9)
EIVLTQSPGTLSLSPGERATLSCRASQSVSSSYLAWYQQKPGQAPRLLIYGASSRATGIPDRESGSGS
GTDFTLTISRLEPEDFAVYYCQQYGESPFTFGPGTKVDIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSS
PVTKSENRGEC
(Variant 3) TNRX-257_GS_KH (HC Het1)
(SEQ ID NO: 91)
EVQLVESGGGVVRPGGSLRLSCCASGFTFDDYGMSWVRRAPGKGLEWVAGINWNGGSTGYADSVKGRF
TISRDNEKNCLYLQMNSLRAEDTALYHCASATSGTAFDIWGQGTMVTVSSPPGGEVQLVQSGAEVKKP
GACVKISCKVSGYTFTRYYMHWVQRAPGKGLEWIGLVDPEDGETIYAEKFQGRVTITADTSTDTAYME
LCSLRSEDTAVYYCATDFVKLDFSLSFQHWGQGTLVTVSSPPGGSGGGGSGGGGSGGQVQLVESGGGV
VQPGRSLRLSCAASGFTESSYGMHWVRQAPGKGLEWVAVIWYDGSNKYYADSVKGRFTISRDNSKNTL
YLQMNSLRAEDTAVYYCAREWAVASWDYGMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKENWY
VDGVEVHNAKTKPREEQYNSTYRVVSVLTVLHQDWLNGKEYKCAVSNKALPAPIEKTISKAKGQPREP
QVCTLPPSREEMTKNQVSLWCLVKGFYPSDIAVEWESNGQPENNYKTTPPVLDSDGSFFLYSKLTVDK
SRWQQGNVFSCSVMHEALHNHYTQKSLSLSPGK
(Variant 3) TNRX-257_GS_KH (HC Het2)
(SEQ ID NO: 92)
QVQLVESGGGVVQPGRSLRLSCAASGFTESSYGMHWVRQAPGKGLEWVAVIWYDGSNKYYADSVKGRE
TISRDNSKNTLYLQMNSLRAEDTAVYYCAREWAVASWDYGMDVWGQGTTVTVSSASTKGPSVFPLAPS
SKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPEAAGGPSVELFPPKPKDTLMISRTPEVTCVVVDVS
HEDPEVKENWYVDGVEVHNAKTKPREEQYNSTYRVVSVLTVLHQDWLNGKEYKCAVSNKALPAPIEKT
ISKAKGQPREPQVYTLPPCREEMTKNQVSLSCAVKGFYPSDIAVEWESNGQPENNYKTTPPVLDSDGS
FFLVSKLTVDKSRWQQGNVFSCSVMHEALHNHYTQKSLSLSPGK
TNRX-257_GS_KH (LC)
(SEQ ID NO: 9)
EIVLTQSPGTLSLSPGERATLSCRASQSVSSSYLAWYQQKPGQAPRLLIYGASSRATGIPDRESGSGS
GTDFTLTISRLEPEDFAVYYCQQYGESPFTFGPGTKVDIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSS
PVTKSENRGEC
(Variant 4) TNRX-257_KH (HC Het1)
(SEQ ID NO: 93)
EVQLVESGGGVVRPGGSLRLSCCASGFTFDDYGMSWVRRAPGKGLEWVAGINWNGGSTGYADSVKGRF
TISRDNEKNCLYLQMNSLRAEDTALYHCASATSGTAFDIWGQGTMVTVSSPPGGEVQLVQSGAEVKKP
GACVKISCKVSGYTFTRYYMHWVQRAPGKGLEWIGLVDPEDGETIYAEKFQGRVTITADTSTDTAYME
LCSLRSEDTAVYYCATDFVKLDESLSFQHWGQGTLVTVSSPPGGQGQGGQGGGQGGGQVQLVESGGGV
VQPGRSLRLSCAASGFTFSSYGMHWVRQAPGKGLEWVAVIWYDGSNKYYADSVKGRFTISRDNSKNTL
YLQMNSLRAEDTAVYYCAREWAVASWDYGMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKENWY
VDGVEVHNAKTKPREEQYNSTYRVVSVLTVLHQDWLNGKEYKCAVSNKALPAPIEKTISKAKGQPREP
QVCTLPPSREEMTKNQVSLWCLVKGFYPSDIAVEWESNGQPENNYKTTPPVLDSDGSFFLYSKLTVDK
SRWQQGNVFSCSVMHEALHNHYTQKSLSLSPGK
(Variant 4) TNRX-257_KH (HC Het2)
(SEQ ID NO: 92)
QVQLVESGGGVVQPGRSLRLSCAASGFTESSYGMHWVRQAPGKGLEWVAVIWYDGSNKYYADSVKGRF
TISRDNSKNTLYLQMNSLRAEDTAVYYCAREWAVASWDYGMDVWGQGTTVTVSSASTKGPSVFPLAPS
SKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPEAAGGPSVELFPPKPKDTLMISRTPEVTCVVVDVS
HEDPEVKENWYVDGVEVHNAKTKPREEQYNSTYRVVSVLTVLHQDWLNGKEYKCAVSNKALPAPIEKT
ISKAKGQPREPQVYTLPPCREEMTKNQVSLSCAVKGFYPSDIAVEWESNGQPENNYKTTPPVLDSDGS
FFLVSKLTVDKSRWQQGNVFSCSVMHEALHNHYTQKSLSLSPGK
TNRX-257_KHI (LC)
(SEQ ID NO: 9)
EIVLTQSPGTLSLSPGERATLSCRASQSVSSSYLAWYQQKPGQAPRLLIYGASSRATGIPDRESGSGS
GTDFTLTISRLEPEDFAVYYCQQYGESPFTFGPGTKVDIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSS
PVTKSENRGEC
(Variant 5) TNRX-257_DS_N297G (HC Het1)
(SEQ ID NO: 94)
EVQLVESGGGVVRPGGSLRLSCCASGFTEDDYGMSWVRRAPGKGLEWVAGINWNGGSTGYADSVKGRF
TISRDNEKNCLYLQMNSLRAEDTALYHCASATSGTAFDIWGQGTMVTVSSPPGGEVQLVQSGAEVKKP
GACVKISCKVSGYTFTRYYMHWVQRAPGKGLEWIGLVDPEDGETIYAEKFQGRVTITADTSTDTAYME
LCSLRSEDTAVYYCATDFVKLDESLSFQHWGQGTLVTVSSPPGGQGQGGQGGGQGGGQVQLVESGGGV
VQPGRSLRLSCAASGFTFSSYGMHWVRQAPGKGLEWVAVIWYDGSNKYYADSVKGRFTISRDNSKNTL
YLQMNSLRAEDTAVYYCAREWAVASWDYGMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTK
VDKKVEPKSCDKTHTCPPCPAPELLGGPSVFCFPPKPKDTLMISRTPEVTCVVVDVSHEDPEVKENWY
VDGVEVHNAKTKPREEQYGSTYRVVSVLTVLHQDWLNGKEYKCKVSNKALPAPIECTISKAKGQPREP
QVYTLPPSREEMTKNQVSLTCLVEGFYPSDIAVEWESNGQPENNYKTTPPVLDSDGSFFLYSDLTVDK
SRWQQGNVFSCSVMHEALHNHYTQESLSLSPGK
(Variant 5) TNRX-257_DS_N297G (HC Het2)
(SEQ ID NO: 95)
QVQLVESGGGVVQPGRSLRLSCAASGFTFSSYGMHWVRQAPGKGLEWVAVIWYDGSNKYYADSVKGRE
TISRDNSKNTLYLQMNSLRAEDTAVYYCAREWAVASWDYGMDVWGQGTTVTVSSASTKGPSVFPLAPS
SKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFCFPPKPKDTLMISRTPEVTCVVVDVS
HEDPEVKFNWYVDGVEVHNAKTKPREEQYGSTYRVVSVLTVLHQDWLNGKEYKCKVSNKALPAPIECT
ISKAKGQPREPQVYTLPPSRKKMTKNQVSLTCLVKGFYPSDIAVEWESNGQPENNYKTTPPVLKSDGS
FFLYSKLTVDKSRWQQGNVFSCSVMHEALHNHYTQKSLSLSPGK
TNRX-257_DS_N297G (LC)
(SEQ ID NO: 9)
EIVLTQSPGTLSLSPGERATLSCRASQSVSSSYLAWYQQKPGQAPRLLIYGASSRATGIPDRESGSGS
GTDFTLTISRLEPEDFAVYYCQQYGESPFTFGPGTKVDIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSS
PVTKSENRGEC

Each of the variants was transiently expressed at 100 mL scale in HEK293 cells, evaluated for titer after 5 days in culture. The molecules were then purified using Protein A chromatography and preparative SEC. The expression and purification characteristics of each TNRX-257 variants are described in Table 5. TNRX-257 and TNRX-257_AA proteins with the charged-based heterodimer motifs had high expression titers and high purity after protein A chromatography (Table 5). The expression of TNRX-257_DS_N297G was roughly ten-fold lower than TNRX-257, suggesting either deglycosylation and/or implementation of the stabilizing disulfide hindered expression (Table 5). All three of the variants harboring the Knobs-in-Holes Fc heterodimerization motif expressed as well as TNRX-257; however, all three demonstrated significantly lower purity after protein A affinity chromatography. Clear evidence of both homodimer and half-antibody (one HC without hinge disulfides) were observed.

TABLE 5
Expression and purity characteristics of the TNRX-257 permutants.

| TNRX-257 | Titer | Protein A Main Peak | Main Peak (%) |
|---|---|---|---|
| permutant | (ug/mL) | (%) | after Prep SEC |
| TNRX-257 | 230 | 90 | 98 |
| TNRX-257_AA | 278 | 87 | 97 |
| TNRX- | 22 | 81 | 99 |
| 257_DS_N297G | | | |
| TNRX-257_KH | 292 | 72 | 84 |
| TNRX- | 312 | 65 | 81 |
| 257_GS_KH | | | |
| TNRX- | 329 | 60 | 77 |
| 257_GS_AA_KH | | | |

The variants were all tested for their ability to activate IL2Rs using human PBMCs that were preactivated with anti-CD3 and anti-CD28 and assessed by cytometry for increased phosphorylation of STAT5 (pSTAT5). All variants except TNRX-257_DS_N297G demonstrated equivalent pSTAT5 EC50s and Emax levels compared to parental TNRX-257 (Table 6), suggesting no loss in activity by modifying the Fc-region for the other variants.

TABLE 6
pSTAT5 IL2R activation of TNRX-257 permutants

| | | pSTAT5 EMAX*10³ |
|---|---|---|
| TNRX-257 | pSTAT5 EC50 | (Mean Fluorescence |
| Permutant | (nM) | Intensity) |
| huIL2 | 0.02 | 108 |
| IgG Control | No increase | 25 |
| TNRX-257 | 0.67 | 61 |
| TNRX-257_AA | 0.82 | 63 |
| TNRX-257_DS_N297G | 1.63 | 61 |
| TNRX-257_KH | 0.72 | 62 |
| TNRX-257_GS_KH | 0.83 | 60 |
| TNRX-257_GS_AA_KH | 0.54 | 62 |

ADDITIONAL SEQUENCES:
IL2RG - IL2RB - LAG3i VH - LAG3 HC Het1
(SEQ ID NO: 48)
EVQLVESGGGVVRPGGSLRLSCCASGFTFDDYGMSWVRRAPGKGLEWVAGINWNGGSTGYADSVKGRF
TISRDNEKNCLYLQMNSLRAEDTALYHCASATSGTAFDIWGQGTMVTVSSPPGGEVQLVQSGAEVKKP
GACVKISCKVSGYTFTRYYMHWVQRAPGKGLEWIGLVDPEDGETIYAEKFQGRVTITADTSTDTAYME
LCSLRSEDTAVYYCATDFVKLDFSLSFQHWGQGTLVTVSSPPGGQGQGGQGGGQGGGQVQLVESGGGV
VQPGRSLRLSCAASGFTFSSYGMHWVRQAPGKGLEWVAVIWYDGSNKYYADSVKGRFTISRDNSKNTL
YLQMNSLRAEDTAVYYCAREWAVASWDYGMDVWGQGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTK
VDKKVEPKSCDKTHTCPPCPAPEAAGGPSVFLFPPKPKDTLMISRTPEVTCVVVDVSHEDPEVKENWY
VDGVEVHNAKTKPREEQYNSTYRVVSVLTVLHQDWLNGKEYKCAVSNKALPAPIEKTISKAKGQPREP
QVYTLPPSREEMTKNQVSLTCLVEGFYPSDIAVEWESNGQPENNYKTTPPVLDSDGSFFLYSDLTVDK
SRWQQGNVFSCSVMHEALHNHYTQESLSLSPGK
LAG3i VH - LAG3 HC Het2
(SEQ ID NO: 49)
QVQLVESGGGVVQPGRSLRLSCAASGFTFSSYGMHWVRQAPGKGLEWVAVIWYDGSNKYYADSVKGRE
TISRDNSKNTLYLQMNSLRAEDTAVYYCAREWAVASWDYGMDVWGQGTTVTVSSASTKGPSVFPLAPS
SKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPEAAGGPSVFLFPPKPKDTLMISRTPEVTCVVVDVS
HEDPEVKENWYVDGVEVHNAKTKPREEQYNSTYRVVSVLTVLHQDWLNGKEYKCAVSNKALPAPIEKT
ISKAKGQPREPQVYTLPPSRKKMTKNQVSLTCLVKGFYPSDIAVEWESNGQPENNYKTTPPVLKSDGS
FFLYSKLTVDKSRWQQGNVFSCSVMHEALHNHYTQKSLSLSPGK
IL2RG - IL2RB - Fc - 6 Res Linker - Het 1
(SEQ ID NO: 50)
EVQLVESGGGVVRPGGSLRLSCCASGFTEDDYGMSWVRRAPGKGLEWVAGINWNGGSTGYADSVKGRF
TISRDNEKNCLYLQMNSLRAEDTALYHCASATSGTAFDIWGQGTMVTVSSGGGSGSEVQLVQSGAEVK
KPGACVKISCKVSGYTFTRYYMHWVQRAPGKGLEWIGLVDPEDGETIYAEKFQGRVTITADTSTDTAY
MELCSLRSEDTAVYYCATDFVKLDESLSFQHWGQGTLVTVSSPPDKTHTCPPCPAPEAAGGPSVELFP
PKPKDTLMISRTPEVTCVVVDVSHEDPEVKENWYVDGVEVHNAKTKPREEQYNSTYRVVSVLTVLHQD
WLNGKEYKCAVSNKALPAPIEKTISKAKGQPREPQVYTLPPSREEMTKNQVSLTCLVEGFYPSDIAVE
WESNGQPENNYKTTPPVLDSDGSFFLYSDLTVDKSRWQQGNVFSCSVMHEALHNHYTQESLSLSPGK
IL2RG - IL2RB - Fc - 4 Res Linker - Het 1
(SEQ ID NO: 51)
EVQLVESGGGVVRPGGSLRLSCCASGFTEDDYGMSWVRRAPGKGLEWVAGINWNGGSTGYADSVKGRF
TISRDNEKNCLYLQMNSLREEDTALYHCASATSGTAFDIWGQGTMVTVSSPPGGEVQLVQSGAEVKKP
GACVKISCKVSGYTFTRYYMHWVQRAPGKGLEWIGLVDPEDGETIYAEKFQGRVTITADTSTDTAYME
LCSLRSEDTAVYYCATDFVKLDESLSFQHWGQGTLVTVSSPPDKTHTCPPCPAPEAAGGPSVELFPPK
PKDTLMISRTPEVTCVVVDVSHEDPEVKENWYVDGVEVHNAKTKPREEQYNSTYRVVSVLTVLHQDWL
NGKEYKCAVSNKALPAPIEKTISKAKGQPREPQVYTLPPSREEMTKNQVSLTCLVEGFYPSDIAVEWE
SNGQPENNYKTTPPVLDSDGSFFLYSDLTVDKSRWQQGNVFSCSVMHEALHNHYTQESLSLSPGK

1. A single immunoglobulin variable domain that binds to IL-2R gamma (IL2-R gamma VH), comprising a framework region of a heavy chain variable region of a human antibody and three complimentary determining regions (CDRs), wherein:
CDR1 comprises an amino acid sequence selected from SEQ ID NO: 10, SEQ ID NO: 11, or SEQ ID NO: 12,
CDR2 comprises an amino acid sequence selected from SEQ ID NO: 13, SEQ ID NO: 14, or SEQ ID NO: 15, and
CDR3 comprises the amino acid sequence of SEQ ID NO: 16.

2. The IL2-R gamma VH of claim 1, comprising the amino acid sequence of SEQ ID NO: 1.

3. A single immunoglobulin variable domain that binds to IL-2R beta (IL2-R beta VH), comprising a framework region of a heavy chain variable region IL2-R beta VH of a human antibody and three complimentary determining regions (CDRs), wherein:
CDR1 comprises an amino acid sequence selected from SEQ ID NO: 17, SEQ ID NO:18, SEQ ID NO: 19, SEQ ID NO: 37, SEQ ID NO: 38, or SEQ ID NO: 39,
CDR2 comprises an amino acid sequence selected from SEQ ID NO: 20, SEQ ID NO:21, or SEQ ID NO: 22, and
CDR3 comprises an amino acid sequence selected from SEQ ID NO: 23, SEQ ID NO: 40, or SEQ ID NO: 41.

4. The IL2-R beta VH of claim 3, comprising the amino acid sequence selected from SEQ ID NO: 2, SEQ ID NO: 35, SEQ ID NO: 36, or SEQ ID NO: 42.

5-13. (canceled)

14. A multi variable domain immunoglobulin comprising the following an amino acid sequence:
[VH1]-[L2]-[VH2]-[L1]-[VH3]-[HCCD1],
wherein:
HCCD1 comprises a first heavy chain constant domain of an antibody comprising a CH1 and a first Fc region (FC1);
VH3 comprises a heavy chain variable domain of an anti-LAG3 antibody;
VH2 comprises one of:
(a) a single immunoglobulin variable domain that binds to IL-2R beta (IL2-R beta VH), comprising a framework region of a heavy chain variable region of a human antibody and three complimentary determining regions (CDRs), wherein:
CDR1 comprises an amino acid sequence selected from SEQ ID NO: 17, SEQ ID NO:18, SEQ ID NO: 19, SEQ ID NO: 37, SEQ ID NO: 38, or SEQ ID NO: 39,
CDR2 comprises an amino acid sequence selected from SEQ ID NO: 20, SEQ ID NO:21, or SEQ ID NO: 22, and
CDR3 comprises an amino acid sequence selected from SEQ ID NO: 23, SEQ ID NO: 40, or SEQ ID NO: 41;
or
(b) single immunoglobulin variable domain that binds to IL-2R gamma (IL2-R gamma VH) comprising a framework region of a heavy chain variable region of a human antibody and three complimentary determining regions (CDRs), wherein:
CDR1 comprises an amino acid sequence selected from SEQ ID NO: 10, SEQ ID NO:11, or SEQ ID NO: 12,
CDR2 comprises an amino acid sequence selected from SEQ ID NO: 13, SEQ ID NO:14, or SEQ ID NO: 15, and
CDR3 comprises the amino acid sequence of SEQ ID NO: 16,
VH1 comprises the other of the IL2-R beta VH or IL2-R gamma VH; and
L1 and L2 are independently optional and may be the same or different amino acid sequence comprising 2-20 amino acids.

15. The multi variable domain immunoglobulin of claim 14, wherein:
VH1 is the IL2-R gamma VH, and
VH2 is the IL2-R beta VH.

16. The multi variable domain immunoglobulin of claim 14, wherein:
VH1 is the IL2-R beta VH, and
VH2 is the IL2-R gamma VH.

17. The multi variable domain immunoglobulin of claim 14, wherein the VH2 comprises SEQ ID NO: 2, SEQ ID NO: 35, SEQ ID NO:36, or SEQ ID NO:42.

18. The multi variable domain immunoglobulin of claim 14, wherein the VH1 comprises SEQ ID NO: 1.

19. The multi variable domain immunoglobulin of claim 14, wherein one of either:
(a) the FC1 comprises SEQ ID NO: 44, SEQ ID NO: 45, SEQ ID NO: 46, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, or SEQ ID NO: 116; or
(b) HCCD1 comprises SEQ ID NO: 4, SEQ ID NO: 5, SEQ ID NO: 43, SEQ ID NO: 96, SEQ ID NO: 97, SEQ ID NO: 98, SEQ ID NO: 99, SEQ ID NO:
100, SEQ ID NO: 101, SEQ ID NO: 102, SEQ ID NO: 103, SEQ ID NO:
104, or SEQ ID NO: 105.

20. The multi variable domain immunoglobulin of claim 14, wherein the VH3 comprises a framework region of a heavy chain variable region of anti-LAG3 antibody and three complimentary determining regions (CDRs), wherein:
CDR1 comprises an amino acid sequence selected from SEQ ID NO: 24, SEQ ID NO: 25, or SEQ ID NO: 26,
CDR2 comprises an amino acid sequence selected from SEQ ID NO: 27, SEQ ID NO: 28, or SEQ ID NO: 29, and
CDR3 comprises the amino acid sequence of SEQ ID NO: 30.

21. The multi variable domain immunoglobulin of claim 14, wherein the VH3 comprises SEQ ID NO: 3.

22. The multi variable domain immunoglobulin of claim 14, further comprising a light chain variable domain of the anti-LAG3-antibody (VL1).

23. The multi variable domain immunoglobulin of claim 22, wherein the VL1 comprises a framework region of a light chain variable region of an anti-LAG3 antibody and three complimentary determining regions (CDRs), wherein;
CDR1 comprises SEQ ID NO: 31,
CDR2 comprises SEQ ID NO: 32, and
CDR3 comprises an amino acid sequence selected from SEQ ID NO: 33, or SEQ ID NO: 34.

24. The multi variable domain immunoglobulin of claim 22, wherein the VL1 comprises SEQ ID NO: 6 or SEQ ID NO: 8.

25. The multi variable domain immunoglobulin of claim 14, further comprising a light chain comprising an amino acid sequence selected from SEQ ID NO: 7 or SEQ ID NO: 9.

26. The multi variable domain immunoglobulin of claim 14, wherein L1 and L2 are independently selected from an amino acid sequence comprising PP, GG, PPGG (SEQ ID NO: 52), GGGSGS (SEQ ID NO:56); GGQGQGGQGGGQGGG (SEQ ID NO: 61), or PPGGQGQGGQGGGQGGG (SEQ ID NO: 68), preferably wherein linker L1 comprises PP, GG, PPGG (SEQ ID NO:52), or GGGSGS (SEQ ID NO: 56), and L2 comprises PP, GGQGQGGQGGGQGGG (SEQ ID NO: 61), or PPGGQGQGGQGGGQGGG (SEQ ID NO: 68).

27. A heterodimeric molecule comprising the multivariable domain immunoglobulin of claim 14 and a second heavy chain constant domain (HCCD2) of an antibody comprising a CH1 and a second Fc region (FC2), wherein FC1 and FC2 heterodimerize.

28. The heterodimeric molecule of claim 27, wherein either:
(a) HCCD1 comprises SEQ ID NO: 5, SEQ ID NO: 97, SEQ ID NO: 99, SEQ ID NO: 101, SEQ ID NO: 103, or SEQ ID NO: 105, when HCCD2 comprises SEQ ID NO:4, SEQ ID NO: 96, SEQ ID NO: 98, SEQ ID NO: 100, SEQ ID NO: 102, or SEQ ID NO: 104, respectively; or
(b) HCCD2 comprises SEQ ID NO: 4, SEQ ID NO: 96, SEQ ID NO: 98, SEQ ID NO: 100, SEQ ID NO: 102, or SEQ ID NO: 104 when HCCD1 comprises SEQ ID NO:5, SEQ ID NO: 97, SEQ ID NO: 99, SEQ ID NO: 101, SEQ ID NO: 103, or SEQ ID NO: 105, respectively.

29. The heterodimeric molecule of claim 27, further comprising a VH3 linked to the N-terminus of HCCD2.

30. The heterodimeric molecule of claim 27, further comprising a light chain variable domain of the anti-LAG3-antibody (VL1).

31. The heterodimeric molecule of claim 30, wherein the VL1 comprises SEQ ID NO: 6 or SEQ ID NO: 8.

32. The heterodimeric molecule of claim 27, further comprising a light chain comprising an amino acid sequence selected from SEQ ID NO: 7 or SEQ ID NO:9.

33-58. (canceled)


SEQUENCE LISTING (116 sequences)

SEQ ID NO: 1 - protein, 118 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSS

SEQ ID NO: 2 - protein, 122 aa, synthetic construct
EVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSS

SEQ ID NO: 3 - protein, 122 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSS

SEQ ID NO: 4 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 5 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 6 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 7 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGESPFTFGPGTKVDIK

SEQ ID NO: 9 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGESPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 10 aa, synthetic construct
GFTFDDYGMS

SEQ ID NO: 11 - protein, 5 aa, synthetic construct
DYGMS

SEQ ID NO: 12 - protein, 7 aa, synthetic construct
GFTFDDY

SEQ ID NO: 13 - protein, 9 aa, synthetic construct
GINWNGGST

SEQ ID NO: 14 - protein, 16 aa, synthetic construct
GINWNGGSTGYADSVK

SEQ ID NO: 15 - protein, 4 aa, synthetic construct
NWNG

SEQ ID NO: 16 - protein, 9 aa, synthetic construct
ATSGTAFDI

SEQ ID NO: 17 - protein, 10 aa, synthetic construct
GYTFTRYYMH

SEQ ID NO: 18 - protein, 5 aa, synthetic construct
RYYMH

SEQ ID NO: 19 - protein, 7 aa, synthetic construct
GYTFTRY

SEQ ID NO: 20 - protein, 8 aa, synthetic construct
LVDPEDGE

SEQ ID NO: 21 - protein, 16 aa, synthetic construct
LVDPEDGETIYAEKFQ

SEQ ID NO: 22 - protein, 5 aa, synthetic construct
DPEDG

SEQ ID NO: 23 - protein, 13 aa, synthetic construct
DFVKLDFSLSFQH

SEQ ID NO: 24 - protein, 10 aa, synthetic construct
GFTFSSYGMH

SEQ ID NO: 25 - protein, 5 aa, synthetic construct
SYGMH

SEQ ID NO: 26 - protein, 7 aa, synthetic construct
GFTFSSY

SEQ ID NO: 27 - protein, 10 aa, synthetic construct
VIWYDGSNKY

SEQ ID NO: 28 - protein, 16 aa, synthetic construct
VIWYDGSNKYYADSVK

SEQ ID NO: 29 - skipped

SEQ ID NO: 30 - protein, 13 aa, synthetic construct
EWAVASWDYGMDV

SEQ ID NO: 31 - protein, 12 aa, synthetic construct
RASQSVSSSYLA

SEQ ID NO: 32 - protein, 7 aa, synthetic construct
GASSRAT

SEQ ID NO: 33 - protein, 9 aa, synthetic construct
QQYGSSPFT

SEQ ID NO: 34 - protein, 9 aa, synthetic construct
QQYGESPFT

SEQ ID NO: 35 - protein, 122 aa, synthetic construct
EVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLPFQHWGQGTLVTVSS

SEQ ID NO: 36 - protein, 122 aa, synthetic construct
EVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLTFQHWGQGTLVTVSS

SEQ ID NO: 37 - protein, 10 aa, synthetic construct
GYTFTDYYMH

SEQ ID NO: 38 - protein, 5 aa, synthetic construct
DYYMH

SEQ ID NO: 39 - protein, 7 aa, synthetic construct
GYTFTDY

SEQ ID NO: 40 - protein, 13 aa, synthetic construct
DFVKLDFSLPFQH

SEQ ID NO: 41 - protein, 13 aa, synthetic construct
DFVKLDFSLTFQH

SEQ ID NO: 42 - protein, 122 aa, synthetic construct
EVQLVQSGAEVKKPGACVKISCKVSGYTFTDYYMHWVQRAPGKGLEWIGLVDPEDGETIYAEKFQGRVTITADTSTDTAYMELCSLRSEDTAVYYCATDFVKLDFSLSFQHWGQGTLVTVSS

SEQ ID NO: 43 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 45 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 46 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 48 - protein, 713 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSSPPGGEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPGGQGQGGQGGGQGGGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 49 - protein, 452 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 50 - protein, 475 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSSGGGSGSEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 51 - protein, 473 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLREEDTALYHCASATSGTAFDIWGQGTMVTVSSPPGGEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 52 - protein, 4 aa, synthetic construct
PPGG

SEQ ID NO: 53 - protein, 19 aa, synthetic construct
PPGGQGQGGQGGGQGGGPP

SEQ ID NO: 54 - protein, 11 aa, synthetic construct
GGQGQGGQGGG

SEQ ID NO: 55 - protein, 5 aa, synthetic construct
GGSGS

SEQ ID NO: 56 - protein, 6 aa, synthetic construct
GGGSGS

SEQ ID NO: 57 - protein, 5 aa, synthetic construct
GGGGS

SEQ ID NO: 58 - protein, 9 aa, synthetic construct
GGGGSGGGS

SEQ ID NO: 59 - protein, 15 aa, synthetic construct
GGGGSGGGGSGGGGS

SEQ ID NO: 60 - protein, 12 aa, synthetic construct
GGGGSGGGGSAS

SEQ ID NO: 61 - protein, 15 aa, synthetic construct
GGQGQGGQGGGQGGG

SEQ ID NO: 62 - protein, 4 aa, synthetic construct
GGQG

SEQ ID NO: 63 - protein, 7 aa, synthetic construct
GGQGQGG

SEQ ID NO: 64 - protein, 7 aa, synthetic construct
PPGGGGS

SEQ ID NO: 65 - protein, 11 aa, synthetic construct
PPGGGGSGGGS

SEQ ID NO: 66 - protein, 17 aa, synthetic construct
PPGGGGSGGGGSGGGGS

SEQ ID NO: 67 - protein, 14 aa, synthetic construct
PPGGGGSGGGGSAS

SEQ ID NO: 68 - protein, 17 aa, synthetic construct
PPGGQGQGGQGGGQGGG

SEQ ID NO: 69 - protein, 6 aa, synthetic construct
PPGGQG

SEQ ID NO: 70 - protein, 9 aa, synthetic construct
PPGGQGQGG

SEQ ID NO: 71 - protein, 13 aa, synthetic construct
PPGGQGQGGQGGG

SEQ ID NO: 72 - protein, 18 aa, synthetic construct
GSTSGSGKPGSGEGSTKG

SEQ ID NO: 73 - protein, 14 aa, synthetic construct
IRPRAIGGSKPRVA

SEQ ID NO: 74 - protein, 5 aa, synthetic construct
GKGGS

SEQ ID NO: 75 - protein, 11 aa, synthetic construct
GKGGSGKGGSG

SEQ ID NO: 76 - protein, 15 aa, synthetic construct
GKGGSGKGGSGKGGS

SEQ ID NO: 77 - protein, 14 aa, synthetic construct
STAGDTHLGGEDFD

SEQ ID NO: 78 - protein, 20 aa, synthetic construct
PPGSTSGSGKPGSGEGSTKG

SEQ ID NO: 79 - protein, 16 aa, synthetic construct
PPIRPRAIGGSKPRVA

SEQ ID NO: 80 - protein, 7 aa, synthetic construct
PPGKGGS

SEQ ID NO: 81 - protein, 13 aa, synthetic construct
PPGKGGSGKGGSG

SEQ ID NO: 82 - protein, 17 aa, synthetic construct
PPGKGGSGKGGSGKGGS

SEQ ID NO: 83 - protein, 16 aa, synthetic construct
PPSTAGDTHLGGEDFD

SEQ ID NO: 84 - protein, 6 aa, synthetic construct
SDKTHT

SEQ ID NO: 85 - protein, 8 aa, synthetic construct
PPSDKTHT

SEQ ID NO: 86 - protein, 713 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSSPPGGEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPGGQGQGGQGGGQGGGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 87 - protein, 452 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 88 - protein, 713 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSSPPGGEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPGGQGQGGQGGGQGGGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 89 - protein, 713 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSSPPGGEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPGGSGGGGSGGGGSGGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 452 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 91 - protein, 713 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSSPPGGEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPGGSGGGGSGGGGSGGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 452 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 93 - protein, 713 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSSPPGGEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPGGQGQGGQGGGQGGGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 713 aa, synthetic construct
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCASATSGTAFDIWGQGTMVTVSSPPGGEVQLVQSGAEVKKPGACVKISCKVSGYTFTRYYMHWVQRAPGKGLEWIGLVDPEDGETIYAEKFQGRVTITADTSTDTAYMELCSLRSEDTAVYYCATDFVKLDFSLSFQHWGQGTLVTVSSPPGGQGQGGQGGGQGGGQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYGSTYRVVSVLTVLHQDWLNGKEYKCKVSNKALPAPIECTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 95 - protein, 452 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYGSTYRVVSVLTVLHQDWLNGKEYKCKVSNKALPAPIECTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 96 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 97 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 98 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 100 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 102 - protein, 330 aa, synthetic construct; identical to SEQ ID NO: 100

SEQ ID NO: 103 - protein, 330 aa, synthetic construct; identical to SEQ ID NO: 101

SEQ ID NO: 104 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYGSTYRVVSVLTVLHQDWLNGKEYKCKVSNKALPAPIECTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 105 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFCFPPKPKDTLMISRTPEVTCVVVDVSHEDPEVKFNWYVDGVEVHNAKTKPREEQYGSTYRVVSVLTVLHQDWLNGKEYKCKVSNKALPAPIECTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 106 - protein, 17 aa, synthetic construct
PPGGSGGGGSGGGGSGG

SEQ ID NO: 107 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 108 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 109 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 111 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 113 - protein, 227 aa, synthetic construct; identical to SEQ ID NO: 111

SEQ ID NO: 114 - protein, 227 aa, synthetic construct; identical to SEQ ID NO: 112

SEQ ID NO: 115 - protein, 227 aa, synthetic construct
DKTHTCPPCPAPELLGGPSVFCFPPKPKDTLMISRTPEVTCVVVDVSHEDPEVKFNWYVDGVEVHNAKTKPREEQYGSTYRVVSVLTVLHQDWLNGKEYKCKVSNKALPAPIECTISKAKGQPREPQVYTLPPSREEMTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 116 - protein, 227 aa, synthetic construct
DKTHTCPPCPAPELLGGPSVFCFPPKPKDTLMISRTPEVTCVVVDVSHEDPEVKFNWYVDGVEVHNAKTKPREEQYGSTYRVVSVLTVLHQDWLNGKEYKCKVSNKALPAPIECTISKAKGQPREPQVYTLPPSRKKMTKNQVSLTCLVKGFYPSDIAVEWESNGQPENNYKTTPPVLKSDGSFFLYSKLTVDKSRWQQGNVFSCSVMHEALHNHYTQKSLSLSPGK